(19) United States
(12) Patent Application Publication
(10) Pub. No.: US 2025/0302768 A1
(43) Pub. Date: Oct. 2, 2025

(54) NANOPARTICLE DOPED POLYETHYLENE GLYCOL BASED GELS AND MEDICAL DEVICES FOR DRUG DELIVERY

(71) Applicant: The General Hospital Corporation, Boston, MA (US)

(72) Inventors: Ebru Oral, Boston, MA (US); Mehmet D. Asik, Boston, MA (US); Orhun Muratoglu, Boston, MA (US)

(21) Appl. No.: 18/864,492

(22) PCT Filed: May 12, 2023
(86) PCT No.: PCT/US2023/066936

§ 371 Date: Nov. 8, 2024

Related U.S. Application Data
(60) Provisional application No. 63/341,386, filed on May 12, 2022.

(51) Int. Cl. A61K 9/51; A61K 31/407; A61K 31/7036; A61K 38/14

(52) U.S. Cl. CPC A61K 9/5161; A61K 9/5192; A61K 31/407; A61K 31/7036; A61K 38/14

(57) ABSTRACT

Provided herein are compositions, kits, and methods of making biodegradable compositions for localized drug delivery. The drug delivery compositions include one or more therapeutic agents that are dispersed within polymerized macromers of the drug delivery composition, loaded within biopolymeric nanoparticles within the drug delivery composition, or both. The release profiles of the one or more therapeutic agents are tunable based on the one or more therapeutic agents for a desired application.

### CROSS REFERENCE TO RELATED APPLICATIONS

This application claims benefit of priority to U.S. Application Ser. No. 63/341,386, filed May 12, 2022, the contents of which is incorporated by reference in its entirety.

### TECHNICAL FIELD

The present invention relates to methods for synthesizing degradable compositions for localized drug deliver with biodegradable macromers doped with nanoparticles. It also relates to methods of incorporating therapeutic agents in these compositions. It also relates to making medical devices comprised of compositions incorporated with therapeutic agents.

### BACKGROUND

Periprosthetic joint infection (PJI) is a serious problem affecting total joint replacement patients. Oral antibiotics can fail in treating persistent bacterial infections, and even the increasing local use of antibiotics incorporated into bone cements has not decreased the incidence of PJI. While systemic drug regimens are the standard in the management of joint replacements, there is an increase in the use of local therapeutic administrations of many therapeutics including analgesics and antibiotics to address pain and infection. The local administration of therapeutics allows for an efficacious dose at a site of injury, surgery, or medical device implantation while decreasing the risk of systemic side effects.

### SUMMARY

The present disclosure provides systems and methods that overcome the aforementioned drawbacks systemic drug regimens via biodegradable compositions, kits, and methods for localized drug delivery of one or more therapeutic agents.

Designing delivery vehicles for therapeutic agents for efficacious local drug delivery is important to achieve and maintain the desired therapeutic effect. While antibiotics are the primary tool in combating infections, combination therapy using multiple antibiotics is commonly desired. In addition, non-conventional antibiotic compounds can also show antibacterial activity on their own as well as additive or synergistic antibacterial activity with commonly used antibiotics. Some of these drugs can require very different dosing for efficacious effect. Similarly, common peri-surgical local cocktails for pain management include analgesics, anti-inflammatoires and other drugs such as vasoconstrictors. To enable and optimize the efficacious local delivery of multiple drugs in the local environment including those with different dosing requirements, there is a need to develop vehicles for controlled release of multiple drugs that may have the same or different release profiles.

Disclosed herein are methods to deliver therapeutic agents, for example antibiotics and commonly used pain medications such as analgesics and non-steroid anti-inflammatory drugs for additive and synergistic antibacterial effect. Also disclosed is a combination drug delivery platform. In a non-limiting example, the combination drug delivery platform includes biodegradable compositions such as (nano)particle-doped (meth)acrylated polyethylene glycol-based gels. The gels are produced by the polymerization of macromers comprising a central moiety, a degradable moiety and cross-linkable moieties. Polymeric or biopolymeric nanoparticles can be loaded into the macromer and formed via an external stimulus into a gel or nanoparticles can be added into already formed gels. Each the nanoparticles, macromer, and gels can be loaded with therapeutic agents. In some examples, micro or macroparticles can be loaded into the macromer or gel in lieu of or in addition to nanoparticles.

According to one aspect of the present disclosure, a method of making a biodegradable composition for localized drug delivery is described. The method comprises polymerizing a mixture of biodegradable macromers; biopolymeric nanoparticles, an initiator, and one or more therapeutic agents, wherein the one or more therapeutic agents are dispersed within the biodegradable macromers, loaded within the biopolymeric nanoparticles, or both dispersed within the biodegradable macromers and loaded within the biopolymeric nanoparticles.

According to another aspect of the present disclosure, a kit for preparing a biodegradable composition for localized drug delivery is described. The kit comprises a first container having biodegradable macromers therein, a second container having biopolymeric nanoparticles therein, and a third container having an initiator therein.

According to another aspect of the present disclosure, a method of preparing a biodegradable composition for localized drug delivery is described. The method comprises mixing the contents of the first container, the second container, and the third container according to the kit described above, polymerizing a mixture, wherein the mixture comprises one or more therapeutic agents dispersed within the biodegradable macromers, loaded within the biopolymeric nanoparticles, or both dispersed within the biodegradable macromers and loaded within the biopolymeric nanoparticles.

According to another aspect of the present disclosure, a mixture is described. The mixture comprises biodegradable macromers; biopolymeric nanoparticles, an initiator, and one or more therapeutic agents, wherein the one or more therapeutic agents are dispersed within the biodegradable macromers, loaded within the biopolymeric nanoparticles, or both dispersed within the biodegradable macromers and loaded within the biopolymeric nanoparticle.

According to another aspect of the present disclosure, a medical device having the mixture according to the preceding paragraph is described.

According to another aspect of the present disclosure, a biodegradable composition for localized delivery comprising a polymerized mixture according to the mixture above is described.

According to another aspect of the present disclosure, a biodegradable composition for localized delivery prepared according to any of the above paragraphs is described.

According to another aspect of the present disclosure, a medical device having the biodegradable composition for localized delivery is described. The medical device includes the biodegradable composition comprising a polymerized mixture according to the above mixture.

According to another aspect of the present disclosure, a medical device having a biodegradable composition for localized delivery prepared according to any of the above paragraphs is described.

These aspects are non-limiting. Other aspects and features of the methods, kits, and compositions described here will be provided below.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure.

FIGS. 1A-1C show release profiles. FIG. 1A shows vancomycin release from MAPLAPEG with vancomycin, CNP & vancomycin, and ketorolac loaded CNP & vancomycin. FIG. 1B shows gentamicin from MAPLAPEG with gentamicin, CNP & gentamicin, and ketorolac loaded CNP & gentamicin. FIG. 1C shows ketorolac from MAPLAPEG with ketorolac loaded CNP, ketorolac loaded CNP & gentamicin, and ketorolac loaded CNP & vancomycin.

FIGS. 2A-2C show cumulative release. FIG. 2A shows vancomycin from MAPLAPEG with vancomycin, CNP & vancomycin, and ketorolac loaded CNP & vancomycin. FIG. 2B shows gentamicin from MAPLAPEG with gentamicin, CNP & gentamicin, and ketorolac loaded CNP & gentamicin. FIG. 2C shows ketorolac from MAPLAPEG with ketorolac loaded CNP, ketorolac loaded CNP & gentamicin, and ketorolac loaded CNP & vancomycin.

### DETAILED DESCRIPTION

In the present disclosure, the embodiments describe methods, compositions, and kits for making biodegradable compositions for localized delivery. The features of the biodegradable drug delivery formulations allow for one or more therapeutic agents to be delivered to a site, and to attain and maintain effective therapeutic concentrations for extended durations of time. Features of the biodegradable drug delivery formulations further allow for controlled and tunable release of the one or more therapeutic agents depending on the selection of components, allowing fora desired treatment plan to be attained. These non-limiting features will be further described below.

These biodegradable composition can be used for the efficacious local delivery of therapeutic agents such as when there is a need for the controlled or tuned release of multiple therapeutic agents. Such tunability may be achieved by selecting appropriate components of the biodegradable composition to achieve a desired drug release profile. Particle doping in a gel can change the drug release profile of a therapeutic agent from in the gel in comparison to the un-doped gel. Therapeutic agent doping in a nanoparticle doped gel can change the drug release profile of a therapeutic agent encapsulated within the nanoparticle in comparison to the un-doped gel. Additionally, the presence of therapeutic agents encapsulated within particles doped in a gel can change the drug release profile of therapeutic agents from the gel in comparison to the un-doped gel or particle doped gel where the particle lacks the encapsulated therapeutic agent. This enabling technology makes the tunable drug release of multiple drugs possible.

By way of example, a non-steroid anti-inflammatory drug (NSAID) may be loaded in nanoparticles and an antibiotic may be loaded in a nanoparticle doped gel or an antibiotic may be loaded in nanoparticles and an NSAID may be loaded in a nanoparticle doped gel. In both cases nanoparticle doping of the gel changes the drug release profile of the therapeutic agent loaded in the gel and in the nanoparticle.

Some macromer and gel preparation methods are described in WO 2017/136726, which is hereby incorporated in its entirety. Some macromer and gel preparation methods are also described in WO 2021/158704, which is hereby incorporated in its entirety. Local delivery vehicles made of the gels described herein can be used in a variety of ways in orthopedic procedures. These gels can be made a priori loaded with drugs in a specific shape including particulates to be applied by themselves, in contact with tissue or in contact with or attached to other implant components. For example, in joint replacement surgery, these gels can be applied onto the non-articulating surfaces of the implant components. In another embodiment, these gels can be applied in contact with internal or external fixation plates in trauma surgeries. In another embodiment, particles made of these gels can be injected in the peri-articular environment. Other implant components can also be prepared and provided coated with gels at the time of the surgery.

The term “drug delivery” refers to the delivery of a drug to a certain tissue or a site in the human body, and the devices used for this purpose are called “drug delivery devices”. Systemic drug delivery methods may produce undesirable side effects and reduce the quantity of drugs reaching the desired site. Various devices and methods have been developed to deliver drugs in a more targeted and efficient manner. The local delivery systems are designed for the controlled release of the encapsulated drugs based on the degradation/swelling of encapsulating media and/or diffusion of the drugs through the device and into the site of interest. While drug delivery devices are designed to control the release profile of the drug, current art is limited in the sequential and tunable delivery of the therapeutic agents.

Nanoparticles are used to encapsulate and/or bind drugs to protect them from degradation, to enable targeting to desired sites and to control release profiles (rate and duration). Spherical nanoparticles have a maximum hydrodynamic diameter of 1000 nm in size, for all other nanoparticles (rods, stars, cages, etc.) are defined by having at least one of the dimensions under 1000 nm. Nanoparticles can encapsulate therapeutic agents during or after particle formation. Ionic, hydrophobic, and hydrophilic interactions can be used to encapsulate therapeutic agents in the nanoparticles formed using ionic gelation, single-emulsion droplet, or double-emulsion droplet techniques. Drugs can also be encapsulated in nanoparticles during particle formation with polymerization-based techniques. Drugs can be loaded onto the particles following formation due to the surface charges and porosity of the particles.

According to an aspect of the present disclosure, a method of making a biodegradable composition for localized drug delivery is described, the method comprises polymerizing a mixture of biodegradable macromers, biopolymeric nanoparticles, an initiator, and one or more therapeutic agents, wherein the one or more therapeutic agents are dispersed within the biodegradable macromers, loaded within the biopolymeric nanoparticles, or both dispersed within the biodegradable macromers and loaded within the biopolymeric nanoparticles.

The term “macromer” refers to a molecule with any molecular weight or a distribution of molecular weights comprising moieties such that several macromers can form new covalent bond(s) with each other and/or with other molecule(s). Macromers are made up of building blocks, the smallest of which is a monomer. Macromonomers are a subset of macromers where the building blocks are the same. In the present invention, macromers are built with different building blocks or moieties.

Macromers may be composed of two biodegradable moieties connected by a central moiety and end capped with two or more cross-linkable moieties such as described in Oral et al. (US 2023/0047214) incorporated here in its entirety. As used herein, the term “moiety” or “chemical moiety” represents a grouping of atoms in a specific arrangement which form covalent chemical bonds in a specific sequence and type. The macromer(s) can be obtained by using microwave radiation from source materials as described. For example, two poly (lactide) with average degree of polymerization of 2 are connected by polyethylene glycol with weight average molecular weight of 200 and then end-capped with two methacrylate groups on each end. In another example, two poly (lactide) with an average degree of polymerization of 4 are connected by polyethylene glycol with weight average molecular weight of 400 and then end-capped with two methacrylate groups on each end. The central moiety can be connected to one or more biodegradable moieties, which can then be connected to one or more cross-linkable moieties. The amount of reactive moieties is not fixed and can be changed. The central moiety can also be connected to non-degradable moieties.

“Connecting moiety” means a molecule or part of molecule that connects biodegradable moiety with biodegradable moiety, biodegradable moiety with cross-linkable moiety, and/or cross-linkable moiety with cross-linkable moiety. A connecting moiety can be chosen from the group of, but are not limited to, polyethylene glycol, polyethylene oxide, polypropylene glycol, 1,6-hexanediol, 2,2,6,6-Tetrakis(hydroxymethyl)cyclohexanol, ethylene glycol, cyanuric acid. Such connecting moieties consist of a mixture of one or more types and consists of a mixture of different molecular weight distributions. The connecting moiety may liquid at room temperature. In one embodiment, the connecting moiety can be a mixture of polyethylene glycol and polypropylene glycol. In another embodiment, the connecting moiety can be a mixture of low molecular weight polyethylene glycol. Low molecular weight polyethylene glycol refers to polyethylene glycol having average molecular weights less than 600 g/mol. In some embodiments, the polyethylene glycol with average molecular weight of 200 g/mol or polyethylene glycol with average molecular weight of 400 g/mol. In another embodiment, the connecting moiety has a random distribution(s) of weight average molecular weight polyethylene glycol. In a preferred embodiment, the connecting moiety can be polyethylene glycol with weight average molecular weight of 200 g/mol (PEG 200). In another preferred embodiment, the connecting moiety can be polyethylene glycol with weight average molecular weight of 400 g/mol (PEG 400).

Biodegradable macromers comprise one or more biodegradable moieties. “Biodegradable moiety” means a molecule or part of molecule that can be degraded (e.g. cleaved and/or destroyed and/or decomposed inside the body) and eliminated by the body. The cleaving, destroying, or decomposing can be through hydrolysis, enzymatic degradation, modification by the liver, excretion by the kidney(s) and/or combinations thereof. Modification by the liver means the changing of the degraded polymer by the liver. Such biodegradable moiety can be but not limited to poly(lactide) (PLA), poly(glycolide) (PGA), poly(epsilon-caprolactone) (PCA), poly(dioxane) (PDA), poly(trimethylene carbonate) (PTMC), and combinations thereof. In one embodiment, the biodegradable moiety is polyglycolide. In another embodiment, the biodegradable moiety is polylactide-co-polyglycolide. In another embodiment, the biodegradable moiety is polytrimethylene carbonate-co-poly(epsilon-caprolactone). In a preferred embodiment, the biodegradable moiety is polylactide with length of 1-8 lactoyl groups. In another preferred embodiment, the biodegradable moiety is polyglycolide with length of 1-8 glycolyl groups. In another preferred embodiment, the biodegradable moiety is polycaprolactone with length of 1-8 epsilon-caprolactone groups. In certain preferred embodiments, the biodegradable moiety is a polylactide with 2-4 lactoyl groups.

As used herein, the term “degradable” or “degradable material” means that the material decomposes through either physical means or chemical means or both physical and chemical means at a certain period of time after the material is implanted as a medical device. By “biodegradation” it is meant to include cleaving, destroying, or decomposing through hydrolysis, enzymatic degradation, biological modification by the liver, excretion by the kidney(s) and combinations of these modes of degradation. Biological modification by the liver means the changing of the chemical structure of the degraded polymer by the liver. As a result, the drug eluting polymer disappears in a certain period after implantation and therefore is no longer a potential surface for colonization by bacteria. The time that it takes for the material to degrade may be as short as one minute or as long as ten years or any length of time between one minute and ten years. The material degradation may be measured by a loss of mass of material, loss of volume of material, decrease in the mechanical stiffness of the material, or change in the molecular structure of the material.

“Cross-linkable moiety” means a molecule or part of a molecule that can form one or more new bond(s) (covalent and/or non-covalent) with another molecule, preferably a macromonomer to create a network of molecule(s) and/or macromonomers. Such cross-linkable moieties can comprise acrylate(s), methacrylate(s), thiols, carboxyls, hydroxyls, amino groups, isocyanates, azides, isothiocyanates, epoxides, and/or combinations thereof). In some embodiment, the cross-linkable moiety comprises acrylate(s), methacrylate(s), or combinations thereof. In more preferred embodiment, the cross-linkable moiety comprises a methacrylate group.

In nonlimiting examples, the biodegradable macromers are methacrylated polyethylene based biodegradable macromers. Methacrylated polyethylene based biodegradable macromers comprise at least one connecting moiety comprising polyethylene glycol, at least one biodegradable moiety, and at least one cross-linkable moiety comprising methacrylate. Methacryalated polyethylene based biodegradable macromers may comprise a central connecting moiety of polyethylene glycol between two biodegradable moieties and two methacrylate cross-linkable moieties.

“Biopolymer” refers to a group of polymer classified into two: naturally occurring and synthetic degradable polymers, including but not limited to glycosaminoglycans, silk, fibrin, polyethylene glycol (PEG), polyhydroxyethyl methacrylate, polyvinyl alcohol, polyacrylamide., Poly (N-vinylpyrrolidone), poly (lactic acid), polyglycolic acid (PGA), polylactic acid-co-glycolic acid (PLGA), poly-e-caprolactone (PCL), polyethylene oxide, propylene polyfumarate (PPF), Polyacrylic acid (PAA), polyhydroxybutyrate, hydrolyzed polyacrylonitrile, polymethacrylic acid, polyethyleneamine, esters of alginic acid; starch acid; and alginate, fully or partially oxidized alginate, hyaluronic acid, carboxy methyl cellulose, heparin, heparin sulfate, chitosan, carboxymethyl chitosan, chitin, purulan, gellan, xanthan, collagen, gelatin, carboxymethyl starch, carboxymethyl dextran, chondroitin sulfate, cationic guar, cationic starch, and combinations thereof.

The term “biopolymeric nanoparticles” refers to nanoparticles comprising a biopolymer. Suitably, the biopolymeric nanoparticle may be composed of alginate, chitosan. elastin, cellulose, chitin, carboxymethyl chitosan, chitosan derivatives, alginate derivatives, polyacrylamides, polyethylene, polylactic acid, poly(lactic-co-glycolic acid), poly amino esters, combinations thereof. The biopolymeric nanoparticles also comprise a cross-linker connecting identical or of different types of chemical groups. The chemical groups may be coupled to one another via covalent, electrostatic, or disulfide interactions, which have a positive or negative charge, which includes one or more of haloformyl or hydroxyl or aldehyde or alkyl or alkenyl or alkynyl or carboxamide, or primary amine or secondary amine or tertiary amine, or azide or azo or benzyl or carbonate ester or carboxylate or carboxyl or cyanate or thiocyanate or disulfide or ether or ester or halo or hydroperoxy, or primary ketimine or secondary ketimine, or primary amine or secondary amine, or imide or isocyanide or isocyanate or isothiocyanate or carbonyl or nitrate or nitryl or nitrosooxy or nitro or nitroso or peroxy or phenyl or phosphino or phosphate or phosphono or pyridyl or sulfide or sulfo or sulfinyl or sulfhydryl groups.

Nanoparticles may be mixed with macromers in an amount to provide a desired drug release profile for one or more therapeutic agent loaded within nanoparticles, the macromers, or both. In some nonlimiting embodiments, the nanoparticles are mixed with the macromers in an amount from about 0.1 to 50 wt %, 1 to 20 wt %, 2 to 20 wt %, or about 2 to 5 wt %. The amount of nanoparticles mixed with the macromers can affect the viscosity of the mixture and one or more additives, such as a viscosity modifier, may optionally be added to the mixture to prepare a mixture having the desired viscosity for the intended application.

The term “initiator” refers to molecule(s) that can initiate polymerization. Said initiator can be activated by light and/or heat and/or chemical means. Upon activation of the initiator, the initiator produces free radicals and/or cationic moieties and/or anionic moieties and interact with macromonomer to initiate polymerization. Said initiator can be activated by external stimuli such as light and/or radiation and/or heat and/or chemical means such as pH or ionic strength changes. Upon providing the external stimulus, the initiator can produce free radicals and/or cationic moieties and/or anionic moieties and interact with the macromer or macromer mixture to initiate its polymerization and/or cross-linking. Initiation can be done by shining ultraviolet light (260-400 nm), blue light (400 nm-500 nm), and/or other visible light (501-800 nm) for a certain period of time and certain radiance. Initiation can be done by heating. Initiating can be done by mixing two or more chemicals. Such initiators can be chosen from the group of but are not limited to benzophenone, 2,2-dimethoxy-2-phenylacetophenone, camphorquinone, ethyl 4-(dimethylamino) benzoate (EDMAB), 2.4.6-trimethylbenzoyldiphenylphosphine oxide, 1-Phenyl-1,2 propanedione, N,N-dimethyl-p-toluidine, Ciba Irgacure® 149, Ciba Irgacure® 184, Ciba Irgacure® 369, Ciba Irgacure® 500, Ciba Irgacure® 651, Ciba Irgacure® 784, Ciba Irgacure® 819, Ciba Irgacure® 907, Ciba Irgacure® 1700, Ciba Irgacure® 1800, Ciba Irgacure® 1850, Ciba Irgacure® 2959, Ciba Darocur® 1173, Ciba Darocur® 4265, Eosin, Rose Bengal, Benzil, Benzoin methyl ether, Isopropoxybenzoin, Benzoin phenyl ether, Benzoin isobutyl ether, Titanocene, benzoyl peroxide, N,N-dimethyl-p-toluidine, and combinations thereof. By light initiation is meant shining ultraviolet (260-400 nm), blue light (400 nm-500 nm), and/or visible light (501-800 nm) for a certain period of time and certain radiance to activate the initiator. By heat initiation is meant adding heat to activate the initiator. By chemical initiation is meant mixing two or more chemicals to activate the initiator. Such initiator can be but not limited to camphorquinone, ethyl 4-(dimethylamino) benzoate (EDMAB), 2.4.6-trimethylbenzoyldiphenylphosphine oxide, 1-Phenyl-1,2 propanedione, N,N-dimethyl-p-toluidine, and combinations thereof. In one embodiment, the initiator is heat sensitive and therefore adding heat to a mixture of macromonomer, initiator, and/or inhibitor, and/or bioactive agent initiates polymerization. In another embodiment, the initiator is light sensitive and therefore shining light of ultraviolet light (260-400 nm), blue light (400 nm-500 nm), and/or visible light (501-800 nm) to a mixture of macromonomer, initiator, and/or inhibitor, and/or bioactive agent initiates polymerization. In another embodiment, the initiator(s) is chemically reactive with each other and therefore mixing the macromonomer, initiator(s), and/or inhibitor, and/or bioactive agent initiates polymerization. An example of an initiator that is heat sensitive is benzoyl peroxide. An example of an initiator that is light sensitive is camphorquinone, ethyl 4-(dimethylamino) benzoate (EDMAB), 2.4.6-trimethylbenzoyldiphenylphosphine oxide, 1-Phenyl-1,2 propanedione. An example of an initiator that is chemically reactive is N,N-dimethyl-p-toluidine, benzoyl peroxide. In a preferred embodiment, the initiator is light activated with blue light (400-500 nm). In more preferred embodiment, the initiator is camphorquinone, ethyl 4-(dimethylamino) benzoate (EDMAB), 2.4.6-trimethylbenzoyldiphenylphosphine oxide, and combinations thereof. In yet another preferred embodiment, the initiator is camphorquinone, ethyl 4-(dimethylamino) benzoate (EDMAB) and combinations thereof.

As used herein, the term “photoinitiator” represents a chemical compound that can produce radical species and/or promote radical reactions when exposed to light irradiation. Common photoinitiators useful in the methods, compositions, and systems described herein include, but are not limited to, benzoin ethers, benzyl ketals, α-diaikoxyacetophenones, ohydroxyalkylphenones, a-amino alkylphenonones, acylphophine oxides, peroxides, and acylphosphinates, azobisisobutyronitrile, 1,1′-azobis(cyclohexanecarbonitrile), di-tert-butyl peroxide, benzoyl peroxide, methyl ethyl ketone peroxide, and acetone peroxide. An exemplary photoinitiator is phenylbis(2,4,6-trimethylbenzoyl) phosphine oxide.

As used herein, the term “thermoinitiator” represents a chemical compound that can produce radical species and/or promote radical reactions when exposed to heat or elevated to a certain temperature. Common classes of thermoinitiators include azo compounds, inorganic peroxides, and organic peroxides. Some non-limiting examples of thermoinitiators include 4,4′-Azobis(4-cyanovaleric acid), 1,1′-Azobis (cyclohexanecarbonitrile), Azobisisobutyronitrile, Ammonium persulfate, Hydroxymethanesulfinic acid, Potassium persulfate, Sodium persulfate, tert-Butyl hydroperoxide, tert-Butyl peracetate, Cumene hydroperoxide, 2,5-Di(tert-butylperoxy)-2, 5-dimethyl-3-hexyne, Dicumyl peroxide, 2,5-Bis(tert-butylperoxy)-2,5-dimethylhexane 2,4-Pentanedione peroxide, 1, 1-Bis(tert-butylperoxy)-3,3,5-trimethylcyclohexane, Benzoyl peroxide, 2-Butanone peroxide, tert-Butyl peroxide, tert-Butyl peroxybenzoate, tert-Butylperoxy 2-ethylhexyl carbonate, tert-Butyl hydroperoxide, and hydrogen peroxide.

The term “therapeutic agent” or “therapeutic” refers to what is known in the art, that is, a chemical substance or a mixture thereof capable of eliciting a healing reaction from the human body. A therapeutic agent can be referred to also as a “drug” or ‘active pharmaceutical ingredient’ (API) in this application. The therapeutic agent can elicit a response that is beneficial for the human or animal. Examples of therapeutic agents are antibiotics, anti-inflammatory agents, anesthetic agents, anticoagulants, hormone analogs, contraceptives, vasodilators, vasoconstrictors, or other molecules classified as drugs in the art. A therapeutic agent can sometimes have multiple functions.

Therapeutic agents may be provided in any suitable form. A therapeutic agents may be provided in solid form, such as drug powders, crystals, or amorphous solids, or liquid form, such as a solution, emulsion, suspension, or dispersion.

Therapeutic agents can be antibiotics such as vancomycin, tobramycin, gentamicin, cefadroxil, cefazolin, cephalexin, cefaclor, cefotetan, cefoxitin, cefprozil, cefuroxime, loracarbef, cefdinir, cefixime, cefoperazone, cefotaxime, cefpodoxime, ceftazidime, ceftibuten, ceftozoxime, ceftriaxone, cefepime, amikacin, streptomycin, doxycycline, erythromycin, gentamicin, isoniazid, rifampin, and ethambutol. Sulfonamides, beta-lactams including penicillin, cephalosporin, and carbepenems, aminoglycosides, quinolones, and oxazolidinones, and metals such as copper, iron, aluminum, zinc, gold, compound, and ions thereof, and various combinations thereof. They can also be chosen from but not limited to such as Gatifloxacin, gemifloxacin, moxifloxacin, levofloxacin, pefloxacin, ofloxacin, ciprofloxacin, aztreonam, meropenem, imipenem, ertapenem, doripenem, piperacillin, Piperacillin-Tazobactam, Ticarcilin-Clavulanic acid, Ticarcillin, ampicillin-sulbactam, amoxicillin-clavulanic acid, ampicillin-amoxicillin, cloxacillin, nafcillin, oxacillin, methicillin, penicillin V, penicillin G, cefpodox, cefdinir, cefditoren, ceftibuten, cefixime, cefuroxime axetil, cefprozil, cefaclor, loracarbef, cephalexin, cefadroxil, cefepime, ceftazidime, ceftaroline, ceftriaxone, ceftizoxime, cefotaxime, cefuroxime, cefuroxime acetil, cefaclor-CD, cefoxitin, cefotetan, cefazolin, cefdinir, cefditoren pivoxil, cefixime, cefpodoxime proxetil, ceftobiprole, colistimethate, linezolid, quinupristin-dalfopristin, metronidazole, rifampin, fosfomycin, nitrofurantoin, TMP-SMX, trimethoprim, fusidic acid, telavancin, teicoplanin, Vancomycin HCl, vancomycin free base, daptomycin, tigecycline, minocycline, doxycycline, telithromycin, clarithromycin, azithromycin, azithromycin ER, erythromycin, clindamycin, chloramphenicol, amikacin, tobramycin, gentamycin, aztreonam, kanamycin, tetracycline, tetracycline HCl, polymyxin B, rifaximin, tigecycline, amphotericin B, fluconazole, itraconazole, ketoconazole, posaconazole, voriconazole, anidulafungin, caspofungin, flucytosine, micafungin, capreomycin, cycloserine, ethambutol, ethionamide, isoniazid, para-aminosalocylic acid, pyrazinamide, rifabutin, rifapentine, streptomycin, albendazole, artemether/lumefantrine, atovaquone, dpasone, ivermectin, mefloquine, miltefosine, nitazoxanide, proguanil, pytimethamine, praziquantel, tinidazole.

Therapeutic agents can also include antivirals such as acyclovir, cidofovir, probenecid, entecavir, famciclovir, foscarnet, ganciclovir, oseltamivir, peramivir, ribavirin, rimantadine, telbiudine, valacyclovir, valgancciclovir, abacavir, atazanavir, darunavir, delaviridine, didanosine, efavirenz, emtricitabine, enfuvirtide, etravirine, fosamprenavir, indinavir, lamivudine, lopinavir, maraviroc, nelfinavir, nevirapine, raltegravir, ritonavir, sasquinavir, stavudine, tenofovir, tipranavir, zidovudine. Antifibrinolytics such as ε-aminocaproic acid, tranexamic acid, lysine, aprotinin. Antineoplastics such as mechlrethamine, phenylalanine mustard, chlorambucil, cyclophosphamide, busulfan, triethylene-thiophosphoramide, carmustine, DTIC, methotrexate, 5-fluorouracil, 6-mercaptopurine, vincristine, procarbazone, prednisone, acivicin, aclarubicin, acodazole, acronine, adozelesin, alanosine, alpha-Tgdr, altretamine, ambomycin, amentantrone acetate, aminopterin, aminothiadiazole, amsacrine, anguinide, aniline mustard, anthramycin, azaribine, 5-aza-2′Deoxycytidine, 8-azaguanine.

Therapeutic agents can be pain management agents such as analgesics, anesthetics, or anti-inflammatory drugs. For a more detailed description of the analgesics, see “Chapter 23—Opioid Analgesics” by Gutstein et al. (pages 569-619) and “Chapter 27—Analgesic-Antipyretic and Anti-inflammatory Agents and Drugs Employed in the Treatment of Gout” by Roberts et al. (pages 687-731), both from Goodman & Gilman's The Pharmacological Basis of Therapeutics, Joel G. Hardman and Lee E. Limbird, eds., 10th Ed., pages 569-619, (2001)) and Glen R. Hanson, “Analgesic, Antipyretic and Anti-Inflammatory Drugs” in Remington: The Science and Practice of Pharmacy, A. R. Gennaro ed. 19th ed., vol. II: 1196-1221(1995). Therapeutic agents can include but are not limited to salicylate, indomethacin, flubiprofen, diclofenac, ketorolac, naproxen, piroxicam, tabferon, ibuprofen, etodolac, nabumetone, tenidap, alcofenac, antipyrine, aminopyrine, dipyrone, aminopyrone, phenyl Butazone, Clofezone, Oxyphenbutazone, Plexazone, Apazone, Benzidoamine, Bucolome, Cinchopen, Clonixin, Ditrazol, Epilizol, Fenoprofen, Floctafenil, Flufenamic acid, Graphenin, Indoprofen, Ketoprofen, Meclofenamic acid, Mephenamine Acid, niflumic acid, phenacetin, salidifamide, sulindac, suprofen, tolmetin and their salts. Salicylates include acetylsalicylic acid, sodium acetylsalicylate, calcium acetylsalicylate, salicylic acid and sodium salicylate. Analgesics include opioid agonist and antagonists. The opioid agonists include but are not limited to alfentanil, allylprodine, alphaprodine, anileridine, benzylmorphine, bezitrnmide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, remifentanil, sufentanil, tilidine, tramadol, pharmaceutically acceptable salts thereof, and mixtures thereof. Other APIs are local anesthetic agents, for example bupivacaine, ropivacaine, dibucaine, procaine, chloroprocaine, prilocaine, mepivacaine, etidocaine, tetracaine, lidocaine, xylocaine, and mixtures thereof. The local anesthetic can be in the form of a salt, for example, hydrochloride, bromide, acetate, citrate, carbonate or sulfate.

The opioid antagonists include but are not limited to naloxone (U.S. Pat. No. 3,254,088, which is incorporated herein by reference in its entirety), naltrexone (U.S. Pat. No. 3,332,950, which is incorporated herein by reference in its entirety) and mixtures thereof; or a pharmaceutically acceptable salt thereof. In still another embodiment, the opioid analgesic or the analgesic is a combination of an opioid agonist and opioid antagonist (examples include, but are not limited to, suboxone which is a combination of buprenorphine and naloxone).

Therapeutic agents can include other compounds with antimicrobial activity such as antimicrobial peptides (AMPs). They can include lipopolysaccharides (LPS), polyguanidines (CPG), bacterial lysates, or defensins.

Therapeutic agents also comprise therapeutic biomolecules, for example, polypeptides, proteins, amino acids, polysaccharides, disaccharides, lipids, natural and synthetic nucleic acids, including but not limited to modified ribonucleic acids (RNA), mRNAs, microRNAs, siRNAs, shRNAs, and other RNAi types, double strand linear deoxyribonucleic acids (DNA), double-strand circular DNA, single strand linear DNA and mixtures thereof.

Any therapeutic agents can be in various chemical forms, such as free base and salts such as hydrochloride sodium, sulfate, acetate, phosphate or diphosphate, chloride, potassium, maleate, calcium, citrate, mesylate, nitrate, tartrate, aluminum, and/or gluconate. For instance, vancomycin hydrochloride, gentamicin sulfate, tobramycin sulfate, and/or polyhexamethylene guanidine phosphate or mixtures thereof. They can also be in ionized or various levels of hydrated forms.

In some embodiments, a first class of therapeutic agent is loaded into the nanoparticles, and a second class of therapeutic agent is loaded into the macromer or gel. In this manner, both therapeutic agents are released from the nanoparticle-doped gels in combination. The release profiles of the different therapeutic agents can be modified for their release to be substantially similar, sequential, at a consistent ratio or showing similar or different rates. Multiple therapeutics can be loaded into nanoparticles and/or the macromer or gel. In some embodiments, one or more therapeutics can be loaded into the macromer or gel and particles can also be loaded in the macromer or gel without additional therapeutics. In this embodiment, the particles are used to modulate the delivery profile of a therapeutic from the gel.

The term “diffusion” refers to what is known in the art; that is, the net movement of molecules from an area of high concentration to an area of low concentration. The term “doping” refers to a general process well known in the art (see, for example, U.S. Pat. Nos. 6,448,315 and 5,827,904), that is introducing additive(s) to a material. “Doping” may be interchangeably used with “loading”. This term can refer to both chemical compounds such as therapeutic agents or polymeric entities such as nanoparticles. Doping may also be done by diffusing an additive into the polymeric material by immersing the polymeric material by contacting the polymeric material with the additive in the solid state, or with a bath of the additive in the liquid state, or with a mixture of the additive in one or more solvents in solution, emulsion, suspension, slurry, aerosol form, or in a gas or in a supercritical fluid. For example, here ‘nanoparticle-doped gels’ are described to indicate the formation of a continuous polymeric material (e.g., gel) with embedded or dispersed nanoparticles as additives. Doping can take place as the polymeric material is being formed such as during the formation of nanoparticles or the curing of gels. The doping process by diffusion can involve contacting a polymeric material, medical implant, or device with an additive, such as vancomycin, for about an hour up to several days, preferably for about one hour to 24 hours, more preferably for one hour to 16 hours. The doping time can be from a second to several weeks, or it can be 1 minute to 24 hours, or it can be 15 minutes to 24 hours in 15 minute intervals. The environment for the diffusion of the additive (e.g., bath, solution, emulsion, paste, slurry and the like) can be heated to room temperature or up to about 200° C. and the doping can be carried out at room temperature or up to about 200° C. For example, when doping a polymeric material by an antioxidant, the medium carrying the antioxidant can be heated to 100° C. and the doping is carried out at 100° C. Similarly, when doping a polymeric material with therapeutic agent(s), the medium carrying the therapeutic agent(s) can be cooled or heated. Or the doping can be carried out at 5, 10, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 230, 240, 250, 260, 270, 280, 290, 300, 320 and 340° C., and any value therebetween. If the additive is a peroxide, the doping temperature may be below the peroxide initiation temperature, at the peroxide initiation temperature or above the peroxide initiation temperature or parts of the doping process may be done at different temperatures. A polymeric material incorporated with an additive is termed an “additive-doped” material. If the additive is a therapeutic agent, a polymeric material incorporated with the additive is termed a “therapeutic agent-doped” polymeric material. Diffusion of additives such as antioxidants are described in Muratoglu et al. (U.S. Pat. Nos. 7,431,874 and 9,370,878), which are incorporated by reference in its entirety.

As used herein, the term “additive” is any chemical compound or mixture of chemical compounds that is intended to improve upon the ease of processing or performance of the final material that is mixed or blended in with the macromer before it is polymerized into a solid gel. Additives may include but are not limited to therapeutic agents, nanoparticles, surfactants, solvents, other monomers, macromers, polymers, acids, bases, salts, ceramics, viscosity modifiers, particles or particulate materials, fibers, organic molecules, or inorganic compounds. When additives are used to dope a material, the additive can be dissolved in a hydrophilic or hydrophobic, polar or non-polar liquid. Therapeutic agents used for doping can be used in powder form. These can be used as supplied or dissolved followed by solvent evaporation to obtain non-solvated forms.

The term “loading capacity” refers to the amount of therapeutic agent in the unit mass of particles or gels. The loading capacity can be between about 1% to about 90%, more preferably 10-30%. For different drugs, the desired release profile can be different. For instance, a slower and extended-release profile might be necessary for a drug, while a short-term of release might be preferable for another drug. In some cases, these drugs can be delivered at the same time or one after the other one, where a dual drug delivery system is required. ‘Dual delivery device’ refers to a drug delivery device, which can deliver two drugs simultaneously. The release profiles of these drugs can be different. The profiles can be modified by the components of the nanoparticle-doped gel system including the type of drugs, the loading capacity of each drug, the size and type of the nanoparticles. In one embodiment of the invention, the hydrophobicity of the gel changes with degradation, which enables fine-tuning of the dual-release profiles. The release profiles of different drugs or therapeutic agents loaded into the gel device can be sequential, which refers to a substantial amount of one drug being released before the another.

The term “polymerization” refers the reaction of monomers or macromers with each other to form covalent bonds and a larger molecule or molecules, which are no longer able to readily react with each other. Said initiating can be through light, heat, and/or chemical means. In one embodiment, the initiator is heat sensitive and therefore adding heat to a mixture of macromer, initiator, and/or inhibitor, and/or bioactive agent initiates polymerization. In another embodiment, the initiator(s) can undergo a spontaneous chemical reaction with one or more of the other additives or the macromer(s); therefore, mixing the macromer(s), initiator(s), and/or inhibitor(s), and/or bioactive agent(s) and/or other additives initiates polymerization. In a preferred embodiment, the initiator is light sensitive and therefore shining light on a liquid, polymerizable mixture; for example, a mixture of macromer(s), initiator(s), and/or therapeutic agent(s), initiates polymerization.

The term “gel” refers to what is known in the art as a form of polymeric material in solid or semi-solid form, which transitions into this form by physical or chemical interactions forming junction points such as crosslinks to prevent fluid-like behavior. Gels can contain liquid and/or solid components. Gels are often categorized by types of interactions causing their formation, for example, ionic, colloidal, organic, or hydrogels. In this application, we describe the use of initiators to induce the formation of gels by using external stimuli such as heating or exposure to light, preferably in a specified wavelength range. During their formation, the described gels can contain liquid, or they do not contain liquid. The components of the gel mixture before the application of the gel-forming stimuli (the ‘pre-gel mixture’) can each be liquid or solid and can form non-solvated or dissolved structures. In a preferred embodiment, the pre-gel mixture contains macromers, initiators, nanoparticles, and therapeutic agent(s). The nanoparticles therein can be loaded with the therapeutic agent(s). Herein, gel and macromer are used interchangeably.

In a non-limiting example, the one or more therapeutic agents are dispersed within the methacrylated polyethylene based biodegradable macromer. For example, any one of an analgesic or antibiotics may be dispersed within the macromer. In another example, an analgesic and antibiotic may be dispersed within the macromer. In another example, one or more analgesics and/or one or more of an antibiotics may be dispersed within the macromer. The therapeutics dispersed within the macromer may be any one or more of the therapeutic agents listed above.

In a non-limiting example, the one or more therapeutic agents are loaded within the biopolymeric nanoparticles. For example, the biopolymeric nanoparticle may be chitosan or alginate. Alternatively, the biopolymeric nanoparticle may be any one of the biopolymeric nanoparticles listed above. In a non-limiting example, an antibiotic, analgesic, or any class of therapeutic agent listed previously may be loaded within the biopolymeric nanoparticle.

In a non-liming example, a first therapeutic agent is dispersed within the methacrylated polyethylene based biodegradable macromers and a second therapeutic agent is loaded within the biopolymeric nanoparticles. In one example, the first and second therapeutic agents may be the same therapeutic agent. Alternatively, the first and second therapeutic agents may be different therapeutic agents.

In a non-limiting example, the mixture of any one of the embodiments described above is polymerized on a medical device surface.

In a non-limiting example, the mixture of any one of the embodiments described above is polymerized on a medical device surface in situ.

In a non-limiting example, the mixture of any one of the embodiments described above is polymerized on the medical device surface at a site of implantation of the medical device.

By “medical device”, what is meant is an instrument, apparatus, implement, machine, implant or other similar and related article intended for use in the diagnosis, treatment, mitigation, cure, or prevention of disease in humans or other animals. An “implantable device” is a medical device intended to be implanted in contact with the human or other animal for a period of time. The primary function of the implantable medical device can be monitoring signals, delivering drugs or the replacement of tissues or the function of tissues among other functions. The implantable medical device can be permanent or can be removed after a period of time. A medical device can be made out of metal, polymer, ceramic or a combination thereof. A medical device can also contain organic tissue or modified organic tissue. Examples of implantable medical devices are acetabular shells, acetabular cups, femoral heads, modular or nonmodular femoral necks, tibial inserts, tibial baseplates, fixation pins, fracture plates, rods, screws, shoulder implants, pacemakers, ventricular assist devices, implantable cardioverter defibrillators, In an embodiment, the medical device is a urinary catheter, a central venous catheter, a femoral central venous catheter. In one embodiment, the medical device can be made of titanium alloy such as TiAI6V4, cobalt chrome alloy, poly ether ether ketone, ultrahigh molecular polyethylene, polyurethane, and combinations thereof. Fixation devices can be used collectively to indicate different components used in the fixation of a fracture, for example fracture plates, fixation pins and screws.

The term “surface” refers to any part of the outside of a solid-form material, which can be exposed to the surrounding liquid, gaseous, vacuum, or supercritical medium. The surface can have a depth into the bulk of the material (normal to the surface planes), from several microns (um) to several millimeters. For example, when a ‘surface layer’ is defined, the layer can have a thickness of several nanometers to several microns (um) to several millimeters. For example, the surface layer can be 100 microns (100 μm) or 500 microns (500 μm) or 1000 microns (1 mm) or 2 mm or it can be between 2 and 5 mm, or any value therebetween. The surface or surfaces can also be defined along the surface planes. For example, a 5 mm wide and 15 mm long oval section of the articulating surface of a tibial knee insert can be defined as a ‘surface’. By “surface of the medical device”, what is also meant is one or more areas on the outside surface(s) of the medical device where the nanoparticle-doped gels can be applied. Surface on the medical device is any area on the medical device exposed to the human, or animal, tissue and/or fluid upon use. In certain embodiments, the surface on the medical device is also modified by chemical and/or physical means. Surface modification can be done by, but not limited to, creating a physical well or a reservoir as a place where the degradable composition can be added. In some embodiments, chemical modification of the surface of the medical device is used to allow for better adhesion of the degradable composition (e.g. by grafting of acrylate, methacrylate, thiol, carboxyl, hydroxyl, amino groups, isocyanates, azides, isothiocyanates, epoxides, and/or a combination thereof on the surface of medical device). In some embodiments, texturing of the surface of the medical device (physically or through chemical etching) is used to provide a surface for the addition of the nanoparticle-doped gel. The surface of the medical implant where the nano-particle-doped gel is applied can be prepared in the factory or in the clinic, operating room, or in the doctor's office.

“Reservoir” means any surface feature that allows at least temporary containment of the liquid, polymerizable liquid. In a preferred embodiment, the medical device has reservoir(s) that are previously machined and the liquid, polymerizable mixture is added into these reservoirs. In another embodiment, reservoir(s) can be formed on the medical device at the time of implantation, for instance in the operating room. In another embodiment, reservoir(s) are created in situ at the time of implantation by medical professionals such as but not limited to physician, nurses, physician assistant. In another preferred embodiment, the reservoirs are pre-formed or formed at the time of implantation on the backside of tibial inserts, sidewalls of tibial insert, rims of acetabular cups, backside of acetabular cups, femoral stems, tibial baseplates, knee femoral components, pacemakers, implantable cardioverter defibrillators, or catheters, in another embodiment, the reservoir is the space above the screw head inside a screw-hole of an acetabular shell or a tibial baseplate.

“Applying on the medical device surface” refers to contacting one or more parts or all the surfaces of a medical device with the nano-particle-doped gel by for example, filling pre-formed reservoirs on the surface(s) of the medical device with mixture before gel formation, painting surface(s) of medical device with mixture, spraying surface of medical device by mixture and combinations thereof. The adhesion between the mixture before gel formation and the medical device surface can be enhanced by using an adhesive. Alternatively, the bond between the medical device surface and the drug-eluting gel on the surface of medical device can be enhanced by mechanical interfacing using design features on the medical device such as pores or locking features. This can also be enhanced by compressing or loading of the drug-eluting gel onto the medical device surface. In one some embodiments, the nanoparticle-loaded gel is added as a layer on one or more reservoir(s) of a medical device and then polymerized. In other embodiments, the nanoparticle-doped mixture before gel formation is added on the surface of a medical device and then polymerized. The polymerizable mixture can be applied to the surface or surface(s) of implantable components such as tissue allograft(s). This application as well as further polymerization can be done before or after the allograft has been implanted. When the polymerizable mixture is not applied to the surface or surfaces of a medical device, it can be applied directly at the site of treatment. Examples for the site of treatment can be the peri-prosthetic tissue around a fracture or a joint implant or degenerative disc(s) or a skin wound.

In one embodiment, the surface(s) include the backside or side walls of the tibial insert or tibial base plate used in total knee replacement. In another embodiment, the surface is along the femoral stem, on the rim of acetabular cup, backside of the acetabular cup as components of total hip replacement. In another implant, the surface is a fixation plate, rods, or screws. In another embodiment, the surface is on the pacemaker pulse generator, inside or outside lumen of catheter.

The term “in situ” refers to what is known in the art as in the intended location. In this context, it refers to the described materials partly being formed in the intended use location. For example, nanoparticle loading into polymerizable mixture with therapeutic agent or the curing of the polymerizable mixture containing macromers, initiator, nanoparticles and therapeutic agent(s) in the intended location. This location can be where treatment is taking place such as in the consultation room, in the clinic, or in the operating room. The location is not limited to any environment for which the treatment is intended. This location can be the site of injury, wound, or implantation of a medical device in the patient.

In a non-limiting example, biodegradable macromers are methacrylated polyethene based biodegradable macromers comprising a biodegradable moiety of repeated lactic acid units, repeated glycolic acid units, or both repeated lactic acid units and glycolic acid units and/or the biopolymeric nanoparticles comprise alginate and/or chitosan. For example, the methacrylated polyethylene based biodegradable macromers comprises a biodegradable moiety of repeated lactic acid units. In another example, the methacrylated polyethylene based biodegradable macromers comprise a biodegradable moiety of repeated glycolic acid units. In these non-limiting examples, the macromer comprise a weight-averaged molecular weight of polyethylene glycol of 200 or 400 g/mol.

In one non-limiting embodiment, the mixture comprises methacrylate-polylactic acid-polyethylene glycol-polylactic acid-methacrylate (MA-PLA-PEG-PLA-MA) macromers, vancomycin dispersed within the MA-PLA-PEG-PLA-MA macromers, and biopolymeric nanoparticles comprising chitosan and ketorolac loaded therein. For example, the MA-PLA-PEG-PLA-MA macromer may be MA-PLA₄-PEG₉-PLA₄-MA.

In another non-limiting embodiment, the mixture comprises MA-PLA-PEG-PLA-MA macromers, gentamicin dispersed within the MA-PLA-PEG-PLA-MA macromers, and biopolymeric nanoparticles comprising chitosan and ketorolac loaded therein. For example, the MA-PLA-PEG-PLA-MA macromer may be MA-PLA₄-PEG₉-PLA₄-MA.

In another non-limiting embodiment, the mixture comprises MA-PLA-PEG-PLA-MA macromers, ketorolac dispersed with the MA-PLA-PEG-PLA-MA macromers, and biopolymeric nanoparticles comprising alginate and chitosan and vancomycin loaded therein. For example, the MA-PLA-PEG-PLA-MA macromer may be MA-PLA₄-PEG₉-PLA₄-MA.

In another non-limiting embodiment, the mixture comprises MA-PLA-PEG-PLA-MA macromers, ketorolac dispersed with the MA-PLA-PEG-PLA-MA macromers, and biopolymeric nanoparticles comprising alginate, chitosan and gentamicin loaded therein. For example, the MA-PLA-PEG-PLA-MA macromer may be MA-PLA₄-PEG₉-PLA₄-MA.

In a non-liming embodiment, the mixture comprises methacrylate-polyglycolic acid-polyethylene glycol-polyglycolic acid-methacrylate (MA-PGA-PEG-PGA-MA) macromers, vancomycin dispersed with the MA-PGA-PEG-PGA-MA macromers, and biopolymeric nanoparticles comprising chitosan, and ketorolac loaded therein. For example, the MA-PGA-PEG-PGA-MA macromer may be MA-PGA₂-PEG₉-PGA₂-MA or MA-PGA₂-PEG_{4.5}-PGA₂-MA.

In another non-limiting embodiment, the mixture comprises MA-PGA-PEG-PGA-MA macromers, gentamicin dispersed with the MA-PGA-PEG-PGA-MA macromers, and biopolymeric nanoparticles comprising chitosan, and ketorolac loaded therein. For example, the MA-PGA-PEG-PGA-MA macromer may be MA-PGA₂-PEG₉-PGA₂-MA or MA-PGA₂-PEG_{4.5}-PGA₂-MA.

In another non-limiting embodiment, the mixture comprises MA-PGA-PEG-PGA-MA macromers, ketorolac dispersed with the methacrylate-polyglycolic acid-polyethylene glycol-polyglycolic acid-methacrylate macromers, and biopolymeric nanoparticles comprising alginate and chitosan, and vancomycin loaded therein. For example, the MA-PGA-PEG-PGA-MA macromer may be MA-PGA₂-PEG₉-PGA₂-MA or MA-PGA₂-PEG_{4.5}-PGA₂-MA.

In another non-limiting embodiment, the mixture comprises MA-PGA-PEG-PGA-MA macromers, ketorolac dispersed with the MA-PGA-PEG-PGA-MA macromers, and biopolymeric nanoparticles comprising alginate and chitosan, and gentamicin loaded therein. For example, the MA-PGA-PEG-PGA-MA macromer may be MA-PGA₂-PEG₉-PGA₂-MA or MA-PGA₂-PEG_{4.5}-PGA₂-MA.

According to another aspect of the present disclosure, a kit for preparing a biodegradable composition for localized drug delivery is described. The kit comprises a first container having biodegradable macromers therein, a second container having biopolymeric nanoparticles therein, and a third container having an initiator therein.

In a non-limiting example, the biodegradable macromers may be biodegradable methacrylated polyethylene based biodegradable macromers as described herein. Likewise, the biopolymeric nanoparticles may be any the nanoparticles described herein. Further, the initiator may be any one of initiators described herein.

In a non-limiting example, the kit further includes one or more therapeutic agents dispersed with the biodegradable macromer, loaded within the biopolymeric nanoparticles, or both dispersed within the biodegradable macromers loaded within the biopolymeric nanoparticles. The one or more therapeutic agents may be any one of the previously described therapeutic agents above.

In a non-limiting example, the one or more therapeutic agents are dispersed within methacrylated polyethylene based biodegradable macromers. For example, any one of an analgesic or antibiotics may be dispersed within the macromer. In another example, an analgesic and antibiotic may be dispersed within the macromer. In another example, one or more analgesics and/or one or more antibiotics may be dispersed within the macromer. The therapeutics dispersed within the macromer may be any one or more of the therapeutic agents listed above.

In a non-limiting example, the one or more therapeutic agents are loaded within the biopolymeric nanoparticles. For example, the biopolymeric nanoparticle may be chitosan or alginate. Alternatively, the biopolymeric nanoparticle may be any one of the biopolymeric nanoparticles listed above. In a non-limiting example, an antibiotic, analgesic, or any class of therapeutic agent listed previously may be loaded within the biopolymeric nanoparticle.

In a non-limiting example, a first therapeutic agent is dispersed within methacrylated polyethylene based biodegradable macromers and a second therapeutic agent is loaded within the biopolymeric nanoparticles. In one example, the first and second therapeutic agents may be the same therapeutic agent. Alternatively, the first and second therapeutic agents may be different therapeutic agents.

According to the aspects of the present disclosure, the kit of any one of the examples described above further includes a fourth container having the one or more therapeutics therein. For example, the fourth container includes one or more therapeutics to be dispersed within the biodegradable macromers

In another non-limiting example, the first therapeutic agent is contained in the fourth container and a second therapeutic agent is loaded within the biopolymeric nanoparticles.

In a non-limiting example, the kit of any one of the examples above further comprises an applicator configured to apply a mixture of biodegradable macromers, biopolymeric nanoparticles, an initiator, and one or more therapeutic agents to a medical device surface, or a medical device, or a radiation source, or any combination thereof.

The term “applicator” refers to a device used to apply the mixture to a surface of a medical device. In a non-limiting example, this may include but is not limited to, a syringe, a brush, a spray nozzle, or a swab.

The term “radiation source” refers to a light source for initiating polymerization. For example, this can be achieved by shining ultraviolet light (260-400 nm), blue light (400 nm-500 nm), and/or other visible light (501-800 nm) for a certain period of time and certain radiance.

In a non-limiting example, the first container, second container, and third container of any one of the examples above are different containers. Alternatively, the first container and second container of any one of the examples above are the same container, and the third container of any one of the examples above is a separate container.

Kits may further comprise instructions for preparing the biodegradable compositions described herein.

### Kit Embodiment 1

In one non-limiting embodiment, the kit comprises first container having methacrylate-polylactic acid-polyethylene glycol-polylactic acid-methacrylate macromers therein, a second container having biopolymeric nanoparticles comprising chitosan therein, and a third container having an initiator therein.

In one example, the first container may have vancomycin dispersed within the methacrylate-polylactic acid-polyethylene glycol-polylactic acid-methacrylate macromers. Alternatively, a fourth container may have vancomycin therein.

In one example, the second container may have ketorolac loaded within the chitosan biopolymeric nanoparticles.

In a non-limiting example, the kit further includes an applicator, or medical device, or radiation source, or any combination thereof.

In a non-limiting example, the first container, the second container, and the third container are different containers. Alternatively, the first container and the second container are the same container, and the third container is a different container.

### Kit Embodiment 2

In one non-limiting embodiment, the kit comprises first container having methacrylate-polylactic acid-polyethylene glycol-polylactic acid-methacrylate macromers therein, a second container having biopolymeric nanoparticles comprising chitosan therein, and a third container having an initiator therein.

In one example, the first container may have gentamicin dispersed within the methacrylate-polylactic acid-polyethylene glycol-polylactic acid-methacrylate macromers. Alternatively, a fourth container may have gentamicin therein.

In one example, the second container may have ketorolac loaded within the chitosan biopolymeric nanoparticles.

In a non-limiting example, the kit further includes an applicator, or medical device, or radiation source, or any combination thereof.

In a non-limiting example, the first container, the second container, and the third container are different containers. Alternatively, the first container and the second container are the same container, and the third container is a different container.

### Kit Embodiment 3

In one non-limiting embodiment, the kit comprises first container having methacrylate-polylactic acid-polyethylene glycol-polylactic acid-methacrylate macromers therein, a second container having biopolymeric nanoparticles comprising alginate and chitosan therein, and a third container having an initiator therein.

In one example, the first container may have ketorolac dispersed within the methacrylate-polylactic acid-polyethylene glycol-polylactic acid-methacrylate macromers. Alternatively, a fourth container may have ketorolac therein.

In one example, the second container may have vancomycin loaded within the alginate and chitosan biopolymeric nanoparticles.

In a non-limiting example, the kit further includes an applicator, or medical device, or radiation source, or any combination thereof.

In a non-limiting example, the first container, the second container, and the third container are different containers. Alternatively, the first container and the second container are the same container, and the third container is a different container.

### Kit Embodiment 4

In one non-limiting embodiment, the kit comprises first container having methacrylate-polylactic acid-polyethylene glycol-polylactic acid-methacrylate macromers therein, a second container having biopolymeric nanoparticles comprising alginate and chitosan therein, and a third container having an initiator therein.

In one example, the first container may have ketorolac dispersed within the methacrylate-polylactic acid-polyethylene glycol-polylactic acid-methacrylate macromers. Alternatively, a fourth container may have ketorolac therein.

In one example, the second container may have gentamicin loaded within the alginate and chitosan biopolymeric nanoparticles.

In a non-limiting example, the kit further includes an applicator, or medical device, or radiation source, or any combination thereof.

In a non-limiting example, the first container, the second container, and the third container are different containers. Alternatively, the first container and the second container are the same container, and the third container is a different container.

### Kit Embodiment 5

In one non-limiting embodiment, the kit comprises first container having methacrylate-polylactic acid-polyethylene glycol-polylactic acid-methacrylate macromers therein, a second container having biopolymeric nanoparticles comprising chitosan therein, and a third container having an initiator therein.

In one example, the first container may have vancomycin dispersed within the methacrylate-polylactic acid-polyethylene glycol-polylactic acid-methacrylate macromers. Alternatively, a fourth container may have vancomycin therein.

In one example, the second container may have ketorolac loaded within the chitosan biopolymeric nanoparticles.

In a non-limiting example, the kit further includes an applicator, or medical device, or radiation source, or any combination thereof.

In a non-limiting example, the first container, the second container, and the third container are different containers. Alternatively, the first container and the second container are the same container, and the third container is a different container.

### Kit Embodiment 6

In one non-limiting embodiment, the kit comprises first container having methacrylate-polylactic acid-polyethylene glycol-polylactic acid-methacrylate macromers therein, a second container having biopolymeric nanoparticles comprising chitosan therein, and a third container having an initiator therein.

In one example, the first container may have gentamicin dispersed within the methacrylate-polylactic acid-polyethylene glycol-polylactic acid-methacrylate macromers. Alternatively, a fourth container may have gentamicin therein.

In one example, the second container may have ketorolac loaded within the chitosan biopolymeric nanoparticles.

In a non-limiting example, the kit further includes an applicator, or medical device, or radiation source, or any combination thereof.

In a non-limiting example, the first container, the second container, and the third container are different containers. Alternatively, the first container and the second container are the same container, and the third container is a different container.

### Kit Embodiment 7

In one non-limiting embodiment, the kit comprises first container having methacrylate-polylactic acid-polyethylene glycol-polylactic acid-methacrylate macromers therein, a second container having biopolymeric nanoparticles comprising alginate and chitosan therein, and a third container having an initiator therein.

In one example, the first container may have ketorolac dispersed within the methacrylate-polylactic acid-polyethylene glycol-polylactic acid-methacrylate macromers. Alternatively, a fourth container may have ketorolac therein.

In one example, the second container may have vancomycin loaded within the alginate and chitosan biopolymeric nanoparticles.

In a non-limiting example, the kit further includes an applicator, or medical device, or radiation source, or any combination thereof.

In a non-limiting example, the first container, the second container, and the third container are different containers. Alternatively, the first container and the second container are the same container, and the third container is a different container.

### Kit Embodiment 8

In one non-limiting embodiment, the kit comprises first container having methacrylate-polylactic acid-polyethylene glycol-polylactic acid-methacrylate macromers therein, a second container having biopolymeric nanoparticles comprising alginate and chitosan therein, and a third container having an initiator therein.

In one example, the first container may have ketorolac dispersed within the methacrylate-polylactic acid-polyethylene glycol-polylactic acid-methacrylate macromers. Alternatively, a fourth container may have ketorolac therein.

In one example, the second container may have gentamicin loaded within the alginate and chitosan biopolymeric nanoparticles.

In a non-limiting example, the kit further includes an applicator, or medical device, or radiation source, or any combination thereof.

In a non-limiting example, the first container, the second container, and the third container are different containers. Alternatively, the first container and the second container are the same container, and the third container is a different container.

According to another aspect of the present disclosure, a method of preparing a biodegradable composition for localized drug delivery is described. The method comprises mixing the contents of the first container, the second container, and the third container according to any one of the kit examples described above, polymerizing a mixture, wherein the mixture comprises one or more therapeutic agents dispersed within the biodegradable macromers, loaded within the biopolymeric nanoparticles, or both dispersed within the biodegradable macromers and loaded within the biopolymeric nanoparticles.

In a non-limiting example, the macromers are biodegradable methacrylated polyethylene based biodegradable macromers as described herein. Likewise, the biopolymeric nanoparticles may be any one of the biopolymeric nanoparticles described herein. Further, the initiator may be any one of the previously described initiators above. Further, the one or more therapeutic agents may be any therapeutic agent described herein.

In a non-limiting example, the one or more therapeutic agents are dispersed within the methacrylated polyethylene based biodegradable macromer. For example, any one of an analgesic or antibiotics may be dispersed within the macromer. In another example, an analgesic and antibiotic may be dispersed within the macromer. In another example, one or more analgesics and/or one or more of an antibiotics may be dispersed within the macromer. The therapeutics dispersed within the macromer may be any one or more of the therapeutic agents listed above.

In a non-limiting example, the one or more therapeutic agents are loaded within the biopolymeric nanoparticles. For example, the biopolymeric nanoparticle may be chitosan or alginate. Alternatively, the biopolymeric nanoparticle may be any one of the biopolymeric nanoparticles listed above. In a non-limiting example, an antibiotic, analgesic, or any class of therapeutic agent listed previously may be loaded within the biopolymeric nanoparticle.

In a non-limiting example, a first therapeutic agent is dispersed within methacrylated polyethylene based biodegradable macromers and a second therapeutic agent is loaded within the biopolymeric nanoparticles. In one example, the first and second therapeutic agents may be the same therapeutic agent. Alternatively, the first and second therapeutic agents may be different therapeutic agents.

According to the aspects of the present disclosure, any one of the examples described above further includes comprises mixing the contents of a fourth container having the one or more therapeutic agents therein with the contents of the first container, the second container, and the third container. For example, the fourth container includes one or more therapeutic agents to be dispersed within the methacrylated polyethylene based biodegradable macromers.

In another non-limiting example, the first therapeutic agent is contained in the fourth container and a second therapeutic agent is loaded within the biopolymeric nanoparticles.

In a non-limiting example of any one of the above examples, the mixture is applied to a medical device surface prior to polymerization of the mixture.

In a non-limiting example, the mixture of any one of the embodiments described above is polymerized on a medical device surface in situ.

In a non-limiting example, the mixture of any one of the embodiments described above is polymerized on the medical device surface at a site of implantation of the medical device.

In a non-limiting example, any one of the mixtures described above is polymerized by irradiating the mixture with ultraviolet radiation.

According to another aspect of the present disclosure, a mixture is described. The mixture comprises biodegradable macromers; biopolymeric nanoparticles, an initiator, and one or more therapeutic agents, wherein the one or more therapeutic agents are dispersed within the biodegradable macromers, loaded within the biopolymeric nanoparticles, or both dispersed within the biodegradable macromers and loaded within the biopolymeric nanoparticle.

In a non-limiting example, the biodegradable methacrylated polyethylene based biodegradable macromers may be any one of the previously described macromers above. Likewise, the biopolymeric nanoparticles may be any one of the previously described nanoparticles above. Further, the initiator may be any one of the previously described initiators above.

In a non-limiting example, the kit further includes one or more therapeutic agents dispersed with the methacrylated polyethylene based biodegradable macromer, loaded within the biopolymeric nanoparticles, or both dispersed within the methacrylated polyethylene based biodegradable macromers loaded within the biopolymeric nanoparticles. The one or more therapeutic agents may be any one of the previously described therapeutic agents above.

In a non-limiting example, the one or more therapeutic agents are dispersed within the methacrylated polyethylene based biodegradable macromer. For example, any one of an analgesic or antibiotics may be dispersed within the macromer. In another example, an analgesic and antibiotic may be dispersed within the macromer. In another example, one or more analgesics and/or one or more of an antibiotics may be dispersed within the macromer. The therapeutics dispersed within the macromer may be any one or more of the therapeutic agents listed above.

In a non-limiting example, the one or more therapeutic agents are loaded within the biopolymeric nanoparticles. For example, the biopolymeric nanoparticle may be chitosan or alginate. Alternatively, the biopolymeric nanoparticle may be any one of the biopolymeric nanoparticles listed above. In a non-limiting example, an antibiotic, analgesic, or any class of therapeutic agent listed previously may be loaded within the biopolymeric nanoparticle.

According to another aspect of the present disclosure, a medical device having the mixture thereon is described. In a non-limiting example, the mixture may by the mixture as previously described.

According to another aspect of the present disclosure, a biodegradable composition for localized delivery comprising a polymerized mixture is described. The mixture may be the mixture as previously described.

According to another aspect of the present disclosure, a biodegradable composition for localized delivery prepared is described. The biodegradable composition may be any one of the examples and embodiments previously described.

According to another aspect of the present disclosure, a medical device having the biodegradable composition for localized delivery is described. The medical device may include the biodegradable composition as described in the immediately preceding paragraph.

According to another aspect of the present disclosure, a medical device having a biodegradable composition for localized delivery prepared according to any of the previous example and embodiments is described.

In a non-limiting example, a macromer and gel preparation method may include microwave treatment. A chemical reaction between a polymeric starting material (central/connecting moiety) and a second monomer is initiated by microwave irradiation. The resultant of this reaction is the block copolymer consisting of the central moiety and the degradable moieties. In one embodiment, the polymeric starting material is mixed with monomer(s) and catalyst(s) and the mixture placed in a microwave oven and exposed to microwave radiation. The duration of microwave treatment can be anywhere between 1 second to several hours or more, in any of the embodiments, the power of the microwave used can be 50W, 60W, 70W, 80W, 90W, 100W, 200W, 300W, 400W, 500W, 600W, 700W, 800W, 900W, 1000W, 1100W, 1200W or more than 1200W or less than 50W or any value in between. The microwave treatment can be carried under atmospheric pressure, under pressures lower than atmospheric pressure, under partial vacuum, with or without active heating, such radiant heating or convection heating. The duration of microwave treatment can be anywhere between 1 second to several hours or more.

Microwave treatment can also be performed on different volumes of mixtures of polymeric starting material and second monomer, in those cases, the duration of exposure to microwave radiation can be modified (made longer or shorter) to homogeneously heat the mixture and initiate the chemical reaction.

The intensity of microwave irradiation may vary throughout the cavity which contains the microwave energy (‘microwave oven’). In such cases, the mixture of polymeric starting material and second monomer may be chosen in specific areas of intensity to increase or decrease the amount of microwave energy absorbed by the mixture of polymeric starting material and second monomer.

In some embodiments, the central/connecting moiety or polymeric starting material is polyethylene glycol (PEG; An) and its molecular weight molecular is about 150-100,000 g/mol but ideally 150-2000 g/mol. In some embodiments, the second monomer B is DL-lactide or L-lactide or D-lactide and its corresponding polymer segment (degradable moiety) is poly (D,L-lactic acid) (PDLLA) or poly (D-lactic acid) (PDLA) or poly (L-lactic acid) (PLLA) and the corresponding block co-polymer is (PDLLA-An-PDLLA) or (PDLA-An-PDLA) or (PLLA-An-PLLA), or the second monomer can be a mixture of these monomers. In some embodiments, the second monomer B is glycolide and its corresponding polymer segment is poly (glycolic acid) (PGA) and the corresponding block co-polymer is (PGA-An-PGA). In one embodiment, the second monomer B is a mixture of lactide and glycolide, and the corresponding random co-polymer is poly (lactic acid-co-glycolic acid) (PLA-r-PGA) and the corresponding block co-polymer is ((PLA-r-PGA)-An-(PLA-r-PGA)). In some embodiments, the number of repeats of the resulting degradable moiety in Bm-An-Bm), i.e., ‘m’ is about 1-20 but ideally 1-10.

The catalyst molecule may be from any of the classes of ring-opening catalysts, including organic catalysts, enzymatic catalysts, or metal catalyst systems. As used herein, the term “catalyst” or “catalyst molecule” represents a chemical compound that drastically accelerates the rate of a chemical reaction without changing the reaction products. For example, stannous octoate is a typical catalyst used for the ring opening polymerization of lactide with polyethylene glycol. Catalysts for the central moiety and degradable moiety reactions initiated by microwave radiation and catalysts for the degradable moiety and cross-linkable moiety reactions can be chosen from the following list but are not limited to it: stannous octoate, 1-(1-Adamantyl)-3-(2,4,6-trimethylphenyl)imidazolinium chloride, Bis(cyclopentadienyl)dimethylzirconium(IV), ichioro[1,3-bis(2,6-isopropylphenyl)-2-imidazolidinylidene](benzylidene)(tricyclohexylphosphine)ruthenium(ll), Dichloro[1,3-Bis (2-methylphenyl)-2-imidazolidinylidene](benzylidene)(tricyclohexylphosphine)ruthenium(ll), Dichloro[1,3-bis(2,4,6-trimethylphenyl)-2-imidazoiidinylidene][3-(2-pyridinyl)propylidene]ruthenium(ll), Diethylmethoxyborane, Dysprosium (ill) trifluoromethanesulfonate, Grubbs Catalyst® C571, Grubbs Catalyst® C711, Grubbs Catalyst® C7827, Grubbs Catalyst® C833, Grubbs Catalyst® C859, Grubbs Catalyst® 1st generation, Grubbs Catalyst® 2nd generation, Grubbs Catalyst® 3rd generation, Hoveyda-Grubbs Catalyst® 1st generation, Hoveyda-Grubbs Catalyst® 2nd generation, Methyltriphenylphosphonium chloride, Neodymium(lll)trifluoromethanesulfonate, or Praseodymium(lll) trifluoromethanesulfonate.

The catalyst can be added to the mixture at concentrations from 0.0001 to 10 wt/wt %, preferably 0,1-3 wt/wt %, most preferably 2 wt/wt %. The catalyst can be dissolved in a solvent; the solvent can be chosen from any of the organic solvents such as acetone, acetonitrile, benzene, butyl acetate, carbon tetrachloride, chloroform, cyclehexane, 1,2 dichloroethane, dichloromethane, dimethylformamide, dimethyl sulfoxide, dioxane, ethanol, ethyl acetate, diethyl ether, heptane, hexane, methanol, methyl-t-butyl ether, 2-butanone, pentane, n-propanol, isopropanol, diisopropyl ether, tetrahydrofuran, toluene, trichloroethylene, water, xylene or no solvent may be used. The amount of solvent in the mixture can be from 1 to 99.9 wt/wt %, preferably 0-5 wt/wt %.

In some embodiments, after the second monomer is reacted with the polymeric starting material to create the block co-polymer, the mixture may be purified by washing with a solvent to remove impurities. Common solvents can be chosen from but are not limited to hexane, diethyl ether, or alcohols such as methanol or ethanol. The washing can be done in multiple steps, each step can use pure solvents or a mixture of solvents. The ratio of block co-polymer to solvent may vary from 1 to 50 volume/volume %, preferably 5-40 volume/volume %, most preferably 10-20 volume/volume %. The temperature of washing may vary from 0 degrees Celsius to 50 degrees Celsius, most preferably 20-30 degrees Celsius. After washing, the solvent may be removed by reduced pressure, elevated temperatures, or a combination of reduced pressure and elevated temperatures. The pressure may range from 20 millibar to 0.5 bar, most preferably 2-5 bar.

The term “washing” refers to procedures used to remove unreacted reaction components from the media. During washing, hydrophobic or hydrophilic solvents can be used, such as water, acetone, acetonitrile, chloroform, hexane, heptane, pentane, ethanol, methanol, propanol, butanol, benzyl alcohol, and other chemicals classified as solvents in the art.

After washing, the co-polymer may be stored at temperatures between 0 and 100 degrees Celsius, under atmospheric conditions or under inert gases such as nitrogen, argon, or any other composition of gases. Most preferably, the co-polymer may be stored at temperatures between 20-30 degrees Celsius and under atmospheric conditions.

In some embodiments, the mixing of the reaction components of the first or second reactions can be done sequentially, or simultaneously. In any of the embodiments, multiple types of the components such as the central moiety or the monomers can be used. Multiple catalysts or solvents can be used.

In any embodiments, the microwave radiation exposure can be performed for a duration of at least 1 second up to several hours, more preferably about 15 seconds to several minutes, most preferable about 30 seconds to 3 minutes, in any of the embodiments, microwave radiation exposure can be performed in a precooled or preheated environment. It can also be performed with active heating or cooling. The heating or cooling can be done at any rate.

The term “heating” refers to bringing a material to a temperature, generally a temperature above that of its current state. It can also refer to maintaining said temperature for a period of time, that is, in some instances, it can be used interchangeably with ‘annealing’. Heating can be done at any rate. The heating rate can be, for example, from 0.001° C./min to 1000° C./min, or any value therebetween, or it can be between 0.1° C./min to 100°° C./min, or it can be from 0.5° C./min to 10° C./min, or it can be any rate from 1° C./min to 50° C./min in 1° C. intervals. The heating can be done for any duration. Heating time can be from 0.1 minutes to 100 years or from 1 minute to 24 hours or from 1 minute to 12 hours, or 30 minutes to 10 hours, or 5 hours, or 6 hours, or 8 hours, or any value therebetween.

The term “cooling” refers to bringing a material to a temperature, generally a temperature below that of its current state. It can also refer to maintaining said temperature for a period of time, that is, in some instances it can be used interchangeably with ‘annealing’. Cooling can be done at any rate. The cooling rate can be from 0.001° C./min to 1000° C./min, or it can be between 0.1° C./min to 100° C./min, or it can be from 0.5° C./min to 10° C./min, or it can be any rate from 1° C./min to 50° C./min in 1° C. intervals, or 2.5° C./min, or any value therebetween. The cooling can be done for any duration. Cooling time can be from 0.1 minutes to 100 years or from 1 minute to 24 hours or from 1 minute to 12 hours, or 30 minutes to 10 hours, or 1 hour, or 2 hours, or 5 hours, or 6 hours, or 8 hours, or any value therebetween.

In some embodiments, the chemical reaction between the reaction product of the first synthesis reaction, that is the block copolymer of the central/connecting moiety and the degradable moieties, and the second monomer may be initiated by microwave irradiation. The resultant of this reaction is the macromer consisting of the central moiety, degradable moieties and cross-linkable moieties. The second monomer can be a precursor that can react with the starting block co-polymer upon an external stimulus to react with the block co-polymer to form the cross-linkable moieties at the chain ends. This external stimulus can be microwave radiation. In some embodiments, the mixture can contain catalyst(s) to change the rate of the reaction.

The duration of the exposure of the mixture to microwave radiation can be anywhere between 1 second to several hours or more. In any of the embodiments, the power of the microwave used can be 50W, 60W, 70W, 80W, 90W, 100W, 200W, 300W, 400W, 500W, 600W, 700W, 800W, 900W, 1000W, 1100W, 1200W or more than 1200W or less than 50W or in between those wattages. The microwave treatment can be carried under atmospheric pressure, under pressures lower than atmospheric pressure, under partial vacuum, with or without active heating, such radiant heating or convection heating.

In some embodiments, the mixing of the reaction components of the first or second reactions can be done sequentially, or simultaneously. In any of the embodiments, multiple types of the components such as the central moiety or the monomers can be used. Multiple catalysts or solvents can be used.

In any embodiments, the microwave radiation exposure can be performed for a duration of at least 1 second up to several hours, more preferably about 15 seconds to several minutes, most preferable about 30 seconds to 3 minutes, in any of the embodiments, microwave radiation exposure can be performed in a precooled or preheated environment, it can also be performed with active heating or cooling. The heating or cooling can be done at any rate.

In some embodiments, the second monomer is methacrylic anhydride or acrylic anhydride and the corresponding chemical moiety is a methacrylate group (MA) or acrylate group (Acr) and the corresponding macromer is (Bn-An-Bn-MA) or (MA-Bn-An-Bn-MA) or (Bn-An-Bn-Acr) or (Acr-Bn-An-Bn-Acr).

In the representations used herein, (e.g. (An), B. (An-Bm-An), (R-An-Bm-An-R), ((An-r-Cp)-Bm-An-(An-r-Cp), and (R-(An-r-CP)-Bm-An-(An-r-C)-R), the capital letters A, B, and C represent monomers. Subscript lowercase letters immediately adjacent to a capital letter indicates the number of repeats of the monomer of that type in a sequence. The capital letter R represents a chemical moiety. Segments or blocks of monomers are set apart by parentheses. Lowercase letters between capital letters and set apart by hyphens indicate an ordering arrangement between the monomers in the sequence. Specifically, “r” indicates that the ordering is random. Hyphens setting apart polymer segments or polymer blocks or chemical moieties indicate that the adjoining polymer segments or blocks or chemical moieties are connected in sequence.

In some embodiments, the macromer may be purified by washing with a solvent to remove impurities. Common solvents can be chosen from but are not limited to hexane, diethyl ether, or alcohols such as methanol or ethanol. The washing can be done in multiple steps, each step can use pure solvents or a mixture of solvents. The ratio of block co-polymer to solvent may vary from 1 to 50 volume/volume %, preferably 5-40 volume/volume %, most preferably 10-20 volume/volume %. The temperature of washing may vary from 0 degrees Celsius to 50 degrees Celsius, most preferably 20-30 degrees Celsius. After washing, the solvent may be removed by reduced pressure, elevated temperatures, or a combination of reduced pressure and elevated temperatures. The pressure may range from 20 millibar to 0.5 bar, most preferably 2-5 bar.

After washing, the co-polymer may be stored at temperatures between 0 and 100 degrees Celsius, under atmospheric conditions or under inert gases such as nitrogen, argon, or any other composition of gases. Most preferably, the co-polymer may be stored at temperatures between 20-30 degrees Celsius and under atmospheric conditions.

In some embodiments, inhibitor molecule(s) or inhibitor molecule(s) or stabilizers or UV absorbers or antioxidants dissolved or mixed with a solvent may be added to or mixed with the macromer to prevent undesirable polymerization or slow the rate of polymerization or prevent oxidation or otherwise improve the stability of the liquid polymerizable macromer. Non-limiting examples of inhibitors include 4-methoxyphenol, 4-Allyloxy-2-hydroxybenzophenone, 2-(2H-Benzotriazol-2-yl)-4,6-bis (1-methyl-1-phenylethyl)phenol, 2-(2H-Benzothazol-2-yl)-4,6-di-tert-pentylphenol, 2-(2H-Benzotriazol-2-yl)-6-dodecyl-4-methylphenol, 2-[3-(2H-Benzotriazol-2-yl)-4-hydroxyphenyl]ethyl methacrylate, 2-(2H-Benzotriazol-2-yl)-4-methyl-6-(2-propenyl)phenol, 2-(2H-Benzotriazol-2-yl)-4-(1,1,3,3-tetramethylbutyl)phenol, 2-(4-Benzoyl-3-hydroxyphenoxy)ethyl acrylate, 3,9-Bis(2,4-dicumylphenoxy)-2,4,8,10-tetraoxa-3,9-diphosphaspiro[5.5]undecane, Bis(octadecyl)hydroxylamine powder, 3,9-Bis(octadecyloxy)-2,4,8,10-tetraoxa-3,9-diphosphaspiro[5.5]undecane, Bis(1-octyloxy-2.2,6,6-tetramethyl 4-piperidyl)sebacate, Bis(2,2;6,6-tetramethyl-4-piperidyl) sebacate, 2-tert-Butyl-6-(5-chloro-2H-benzotriazol-2-yl)-4-methylphenol, 2-tert-Butyl-4-ethylphenol, 5-Chloro-2-hydroxybenzophenone, 5-Chloro-2-hydroxy-4-methylbenzophenone, 2,4-Di-tert-butyl-6-(5-chloro-2H-benzotriazol-2-yl)phenol, 2,6-Di-tert-butyl-4-(dimethylaminomethyl)phenol, 3′,5′-Dichloro-2′-hydroxyacetophenone, Didodecyl 3,3′-thiodipropionate, 2,4-Dihydroxybenzophenone, 2,2′-Dihydroxy-4-methoxybenzophenone, 2′,4-Dihydroxy-3′-propylacetophenone, 2,3-Dimethylhydroquinone, 2-(4,6-Diphenyl-1,3,5-triazin-2-yl)-5-[(hexyl) oxy]-phenol, 5-Ethyl-1-aza-3,7-dioxabicydo[3.3.0]octane, Ethyl 2-cyano-3,3-diphenylacrylate, 2-Ethylhexyl cyano-3,3-diphenylacrylate, 2-Ethylhexyl trans-4-methoxycinnamate, 2-Ethylhexyl salicylate, 2-Hydroxy-4-(octyloxy)benzophenone, Menthyl anthranilate, 2-Methoxyhydroquinone, Methyl-p-benzoquinone, 2,2′-Methylenebis[6-(2H-benzotriazol-2-yl)-4-(1,1,3,3-tetramethylbutyl)phenol], 2,2-Methylenebis(6-tert-butyl-4-ethylphenol), 2.2,-Methylenebis(6-tert-butyl-4-methylphenol), Methylhydroquinone, 4-Nitrophenol sodium salt hydrate, Octadecyl x3-(3,5-di-tert-butyl-4-hydroxyphenyl)propionate, Pentaerythritol tetrakis(3,5-di-tert-butyl-4-hydroxyhydrocinnamate), 2-Phenyl-5-benzimidazolesulfonic acid, Poly[[6-[(1,1,3,3-tetramethylbutyl)amino]-s-triazine-2,4-diyl]-[(2,2,6,6-tetramethyl-4-piperidyl)imino]-hexamethylene-[(2,2,6,6-tetramethyl-4″piperidyl)imino], Sodium D-isoascorbate monohydrate, Tetrachloro-1,4-benzoquinone, Triisodecyl phosphite, 1, 3, 5-Trimethyl-2,4, 6-tris{3, 5-d i-tert-butyl-4-hydroxybenzyl)benzene, Tris(4-tert-butyl-3-hydroxy-2,6-dimethylbenzyl) isocyanurate, Tris (2,4-di-tert-butylphenyl) phosphite, and 1,3,5-Tris(2-hydroxyethyl)isocyanurate, Tris(nonylphenyl) phosphite. The solvent can be aqueous or organic or a mixture. The inhibitor molecule(s) can be dissolved in the liquid, polymerizable mixture or the macromer(s) or the inhibitor can be partially dissolved or the inhibitor may be largely immiscible. The inhibitor can be mixed with the liquid, polymerizable mixture such that the resulting concentration of the inhibitor is from 1 ppm to 10 wt/wt %, or 10 ppm to 1 wt/wt %, more preferably 100 ppm to 1000 ppm.

In some embodiments, the inhibitor molecule is directly blended with the polymeric material by stirring, shaking, physically mixing, or sonication, or using a machine apparatus to mix, shake, stir, or otherwise disperse the initiator molecule.

In any of the embodiments, the macromer or any material or device made from or partially made from the polymerized macromer may be sterilized for use as an implantable medical device. Common methods of sterilization are included but not limited to treatment with ethylene oxide gas, electron-beam radiation, gamma radiation treatment with peroxides or autoclaving.

The term “sterile” refers to a condition of an object, for example, an interface or a hybrid material or a medical implant containing interface(s), wherein the interface is sufficiently sterile to be medically acceptable, i.e., will not cause an infection or require revision surgery. The object, for example a medical implant, can be sterilized using ionizing radiation or gas sterilization techniques. Gamma sterilization is well known in the art. Electron beam sterilization is also used. Ethylene oxide gas sterilization and gas plasma sterilization are also used. Autoclaving is another method of sterilizing medical implants. Exposure to solvents or supercritical fluids for sufficient to kill infection-causing microorganisms and/or their spores can be a method of sterilizing.

In any embodiments, the blending of the therapeutic agent and/or initiator molecule can be aided by the addition of additives such as surfactants, solvents, or compatibilizers.

In some embodiments, the initiator molecule or source of light radiation or temperature used to initiate the polymerization reaction may be changed to maintain compatibility with a therapeutic agent. For example, ketorolac tromomethane inhibits the polymerization reaction at an irradiation wavelength of 365 nm, but a light source of 395 nm successfully initiates the polymerization reaction. In some embodiments, the therapeutic agent is not compatible with elevated temperature, so a different initiator may be selected. in some embodiments, the amount of drug mixed with the macromer may be increased or decreased to increase or decrease the rate of release of the therapeutic agent. Drugs or therapeutic agents or additives can be mixed at a ratio of 0.0001 to 99 wt % of the liquid polymerizable mixture, more preferably at a ratio of 0.1 to 20wt %, most preferably at a ratio of 1 to 10 wt %.

In some embodiments, the structure of the macromer may be changed in order to increase or decrease the rate of release of the therapeutic agent, or multiple macromers with different structures can be blended to increase or decrease the rate of release of the therapeutic agent.

In some embodiments, multiple therapeutic agents may be mixed into the liquid polymerizable mixture, In some embodiments, the addition of a second therapeutic may increase or decrease the rate of release of the first therapeutic agent, and the first therapeutic agent may increase or decrease the rate of release of the first therapeutic agent.

In some embodiments, the shape or size or surface area of the final material may be modified to increase or decrease the rate of release of the loaded therapeutic agent or therapeutic agents.

In some embodiments, the liquid polymerizable mixture with additives can be applied to another surface such as a biological tissue, a polymeric material, a metal or a ceramic or hybrid materials. The application can be in one step before polymerization or layers can be applied and polymerized on each other. Each of these layers can contain one additive or multiple additives. These additives in the different layers can be the same or different. The thickness of each layer can be several microns to several millimeters, the entire thickness of the polymerized layer on the second surface can be microns to 10 millimeters. The thickness can be uniform or can be modified as desired along the applied surface(s).

In some embodiments, the initiator molecule is incorporated on the application surface placed in contact with the liquid polymerizable mixture. In these embodiments, the polymerization is initiated at the interface between the polymeric material and the application surface. The application surface may be porous, contain holes, or other such geometric features. In some embodiments the liquid polymerizable mixture may infiltrate the holes or pores of the application surface and the polymerization may be initiated such that the holes or pores are filled or partially filled by the resulting polymerized solid gel.

In some embodiments, the macromer can be spread as a thin layer on top of an application surface or device, then the polymerization can be initiated to produce an application surface or device covered with a layer of solid gel.

In some embodiments, the solid gel formed from the polymerized macromer mixture may be physically cut, re-shaped, or machined to produce a solid gel with a different two-dimensional or three-dimensional shape. Other geometric features may be created as the result of cutting or re-shaping or machining, including but not limited to holes, indentations, tapered holes, blunt holes, or screw holes.

In some embodiments, the macromer can be polymerized inside of a container with a specific shape or size. The solid gel will then take the shape of the container.

In some embodiments, the thickness of the layer of solid gel may be modified to ensure a fully-polymerized material is created.

As used herein, the term “polymer segment” means and includes a grouping of multiple monomer units of the same type (i.e. a homopolymer segment) or of different types (i.e. a co-polymer segment) of constitutional units joined together into a continuous polymer chain.

As used herein, the term “polymer block” means and includes a grouping of multiple monomer units of the same type (i.e. a homopolymer block) or of different types (i.e. a co-polymer block) of constitutional units joined together into a continuous polymer chain that forms part of a larger polymer of even greater length.

As used herein, the term “block co-polymer” means and includes a polymer composed of chains where each chain is composed of two or more polymer blocks as defined above. A block co-polymer may be represented herein by (An-Bm), where A and B represent monomers and n and m each represent the number of repeats. As used herein, the term “random co-polymer” means and includes a polymer chain formed from two different monomers arranged in a pattern having no particular order to form a polymer segment. Random co-polymers may be represented by (An-r-Cp), where the capital letters A and C represent monomers, n and p each represent the number of repeats, and r represents that the sequence of A and C monomers is random and has no particular order.

As used herein, the term “microwave radiation” represents electromagnetic radiation with wavelengths ranging from one meter to one millimeter. Microwave irradiation can be done at temperatures between 0 and 100 degrees Celsius, but preferably between 20 and 30 degrees Celsius, it can be done in inert atmosphere or in environments with varying concentrations of gases such as nitrogen or oxygen or mixtures thereof, By ‘microwave oven’ is meant an appliance that contains a cavity into which microwaves are sent, causing items in the cavity to be irradiated with microwave radiation. The power of the microwave used can be 50 W, 60 W, 70 W, SOW, 90 W, 100 W, 200 W, 300 W; 400 W, 500 W, 600 W, 700 W, 800 W, 900 W, 1000 W, 1100 W, 1200 W or more than 1200 W or less than 50 W or in between those wattages. The microwave treatment can be carried under atmospheric pressure, under pressures lower than atmospheric pressure, under partial vacuum, with or without active heating, such radiant heating or convection heating. The intensity of microwave radiation may be homogeneous throughout the cavity, or certain locations within the cavity may experience higher intensity of radiation than others. The cavity may be designed to intentionally provide uniform or non-uniform intensity of microwave radiation by changing the shape of the cavity or the source of microwave radiation or by adding elements to the cavity that reflect microwave radiation in a specific direction or directions.

The terms “about” or “approximately” in the context of numerical values and ranges refers to values or ranges that approximate or are close to the recited values or ranges such that the invention can perform as intended, such as utilizing a method parameter (e.g., time, dose, dose rate/level, and temperature), having a desired amount of antibiotics, desired degree of cross-linking and/or a desired lack of or quenching of free radicals, as is apparent to the skilled person from the teachings contained herein. This is due, at least in part, to the varying properties of polymer compositions. Thus, these terms encompass values beyond those resulting from systematic and random error. These terms make explicit what is implicit, as known to the person skilled in the art.

While present inventive concepts have been described with reference to particular embodiments, those of ordinary skill in the art will appreciate that various substitutions and/or other alterations may be made to the embodiments without departing from the spirit of present inventive concepts.

Accordingly, the descriptions provided herein are meant to be exemplary and does not limit the scope of present inventive concepts.

A number of examples are provided herein. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the present inventive concepts.

### EXAMPLES

### Example 1

Methacrylated poly(lactic acid-co-ethylene glycol) (MAPLAPEG) macromers were synthesized with microwave synthesis with a lactate degree of polymerization (DP) of 4 and PEG400 (DP 9.5). Chitosan nanoparticles (CNP) were synthesized using ionic gelation and ketorolac tromethamine was loaded into the CNPs. For polymerization, the MAPLAPEG macromers were mixed with 10 mg/mL Phenylbis(2,4,6-trimethylbenzoyl) phosphine oxide initiator at a ratio of 1 μL each 20 μL of macromer and exposed to UV radiation at 395 nm for two minutes to form a 250 mg gel strip with the dimensions of 5 mm×3 mm×20 mm. The test groups were formed as follows: (1) gel and antibiotic; (2) gel loaded with antibiotic and CNPs; (3) gel loaded with antibiotic and (4) gel and ketorolac loaded CNPs. Gels loaded with only ketorolac loaded CNPs were used as controls for ketorolac elution. Cured gels were placed in 5 ml phosphate buffered saline and samples were collected at 0.25, 1, 2, 3, 4, 7, 14, 21, and 28-day time points. Eluent concentrations were measured using UV visible spectrometry. Gentamicin sulfate samples were treated with O-phthalaldehyde and the fluorescence was measured after complexation [4].

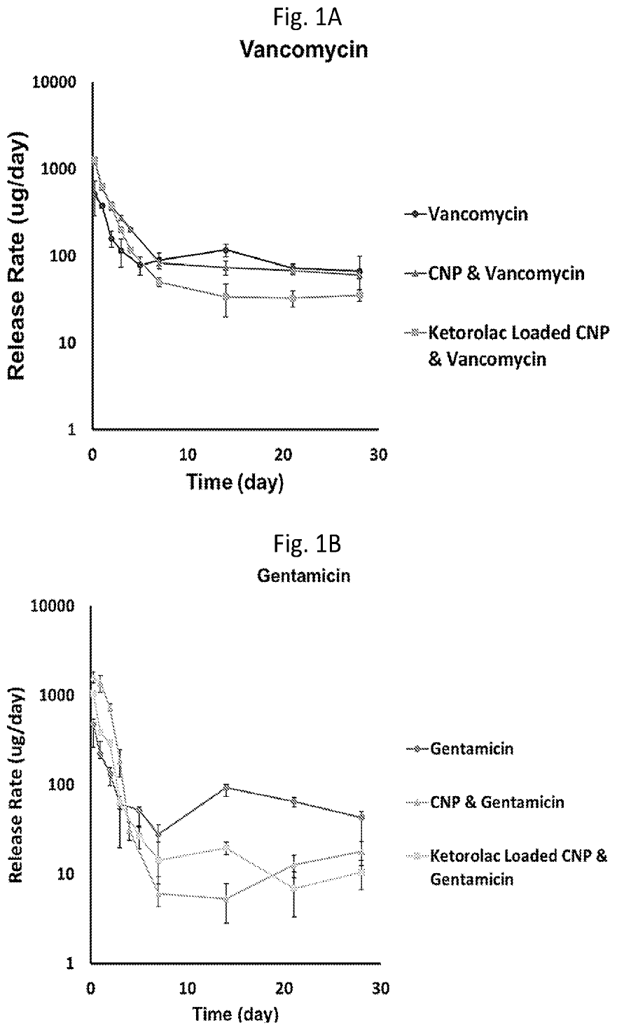

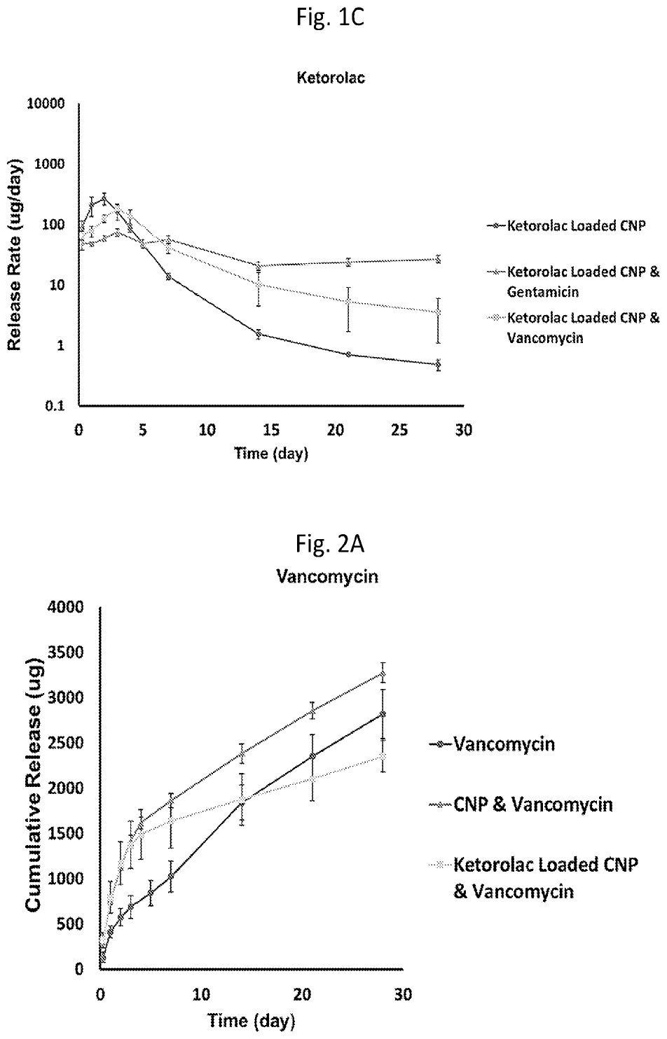

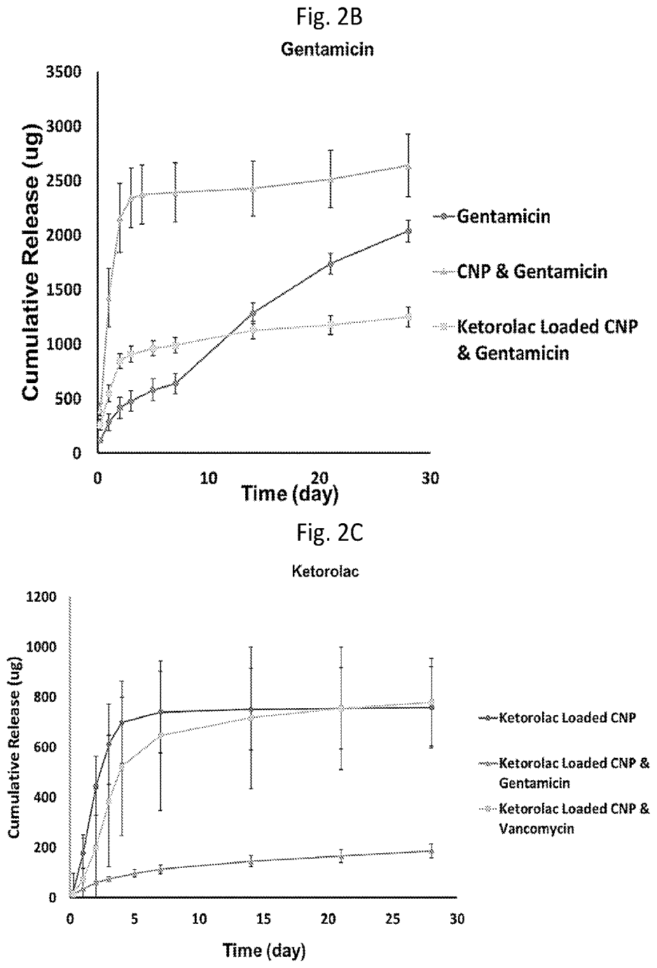

FIGS. 1A-1C depict the release rates of vancomycin, gentamicin and ketorolac from the gels loaded only with antibiotics and those loaded also with particles and those loaded with particles containing ketorolac. FIGS. 2A-2C illustrates the cumulative release of vancomycin, gentamicin, and ketorolac. The sample size for each group was four and standard deviation was calculated for each sample group.

The gentamicin and vancomycin concentrations eluted from MAPLAPEG polymers were maintained above the minimum inhibitory concentration (MIC) values for 4 weeks (0.5-2 ug/ml for Vancomycin and 1.5-3 ug/ml for Gentamicin for Staphylococcus aureus). The release profiles of both antibiotics and ketorolac from CNP loaded MAPLAPEG gels were different from those of controls, suggesting that release profiles could be altered by CNP loading and by the interactions of the two therapeutics loaded into the dual-delivery vehicle.

These gels loaded with particles can release multiple therapeutics at desired concentrations and can potentially be applied in situ in the operating room. One advantage is their high loading efficiency as they are prepared by mixing with powder drugs for local applications during the surgery. These dual-delivery vehicles are promising for improving the local delivery of therapeutics in orthopedics.

### Example 2

Sequential dual drug release from chitosan nanoparticle doped MAPLA4-PEG9-PLA4-MA gels

### Synthesis of MA-PLA4-PEG9-PLA4-MA

3 g of PEG with a molecular weight of 400 g/mol was mixed with 4.32 g of DL lactide and 60 ul of stannous octoate. The mixture was heated with microwave radiation for 2 minutes to produce PLA4-PEG9-PLA4. 2.5 ml of methacrylic anhydride was added and the mixture was heated with microwave radiation for 2 minutes to produce MA-PLA4-PEG9-PLA4-MA. Later the macromers were washed using both hydrophobic solutions and hydrophilic solutions. 70 uL mequinol was added to the macromers.

Synthesis of ketorolac loaded chitosan nanoparticles

4 ml of 0.3% Tripolyphosphate solution was added drop wise to 12 ml of 0.5% Chitosan Solution in 1% Acetic Acid at pH=4.5 on a magnetic stirrer. The mixture was left overnight or at least 8 hours stirring for homogenization. The mixture then transferred to high-speed centrifuge tubes and centrifuged with 20,000×g speed and washed with distilled water three times by removing the supernatants and refreshing it with distilled water. After the particles have finished washing, excess water is removed and synthesized particles dispersed in at least 1 ml Ketorolac solution (0-30 mg, 10 mg is preferred). The mixture was placed on a shaker for 2 hours at room temperature. After 2 hours, at least 1 ml of distilled water was added to the mixture and centrifuged at high speed for washing. The particles were washed three times at 20,000×g by discarding the supernatant and adding 5 ml of distilled water. After washing, particles were dispersed in water and were frozen at −20° C. The frozen particles were lyophilized at least for 24 hours to remove water.

Gel preparation for dual drug delivery
Gel preparation for vancomycin and ketorolac delivery with chitosan nanoparticles

0-10 mg of ketorolac loaded chitosan nanoparticle powder and 0-10 mg of vancomycin powder was mixed in a vial with 250 mg of MA-PLA4-PEG9-PLA4-MA of 10 mg/mL Phenylbis (2,4,6-trimethylbenzoyl) phosphine oxide initiator at a ratio of 1 μL each 20 μL of macromer and exposed to UV radiation at 395 nm for two minutes to form a 250 mg gel strips.

Gel preparation for gentamicin and ketorolac delivery with chitosan nanoparticles

0-10 mg of ketorolac (tromethamine) loaded chitosan nanoparticle powder and 0-10 mg of gentamicin (sulfate) powder was mixed in a vial with 250 mg of MA-PLA₄-PEG₉-PLA₄-MA of 10 mg/mL Phenylbis(2,4,6-trimethylbenzoyl) phosphine oxide initiator at a ratio of 1 μL each 20 μL of macromer and exposed to UV radiation at 395 nm for two minutes to form a 250 mg gel strips.

### Example 3

Sequential dual drug release from chitosan nanoparticle doped MA-PGA₂-PEG₉-PLA₂-MA gels
Synthesis of MA-PGA₂-PEG₉-PGA₂-MA

3 g of PEG with a molecular weight of 400 g/mol was mixed with 1.72 g of DL glycolide and 75 ul of stannous octoate. The mixture was heated with microwave radiation for 2 minutes to produce PGA₂-PEG₉-PGA₂. 2.5 ml of methacrylic anhydride was added and the mixture was heated with microwave radiation for 2 minutes to produce MA-PGA₂-PEG₉-PGA₂-MA. Later, the macromers were washed using hydrophobic solutions and hydrophilic solutions. 48 ul mequinol was added to the macromers.

Synthesis of ketorolac loaded chitosan nanoparticles

4 ml of 0.3% Tripolyphosphate solution was added drop wise to 12 ml of 0.5% Chitosan Solution in 1% Acetic Acid at pH=4.5 on a magnetic stirrer. The mixture was left overnight or at least 8 hours stirring for homogenization. The mixture then transferred to high-speed centrifuge tubes and centrifuged with 20,000×g speed and washed with distilled water three times by removing the supernatants and refreshing it with distilled water. After the particles have finished washing, excess water is removed and synthesized particles dispersed in at least 1 ml ketorolac tromethamine solution (0-30 mg, 10 mg is preferred). The mixture was placed on a shaker for 2 hours at room temperature. After 2 hours, at least 1 ml of distilled water was added to the mixture and centrifuged at high speed for washing. The particles were washed three times at 20,000×g by discarding the supernatant and adding 5 ml of distilled water. After washing, particles were dispersed in water and froze at −20° C. The frozen particles were lyophilized at least for 24 hours to remove water.

Gel preparation for dual drug delivery
Gel preparation for vancomycin and ketorolac delivery with chitosan nanoparticles

0-10 mg of ketorolac tromethamine loaded chitosan nanoparticle powder and 0-10 mg of vancomycin hydrochloride powder was mixed in a vial with 250 mg of MA-PGA₂-PEG₉-PGA₂-MA of 10 mg/mL Phenylbis (2,4,6-trimethylbenzoyl) phosphine oxide initiator at a ratio of 1 μL each 20 μL of macromer and exposed to UV radiation at 395 nm for two minutes to form a 250 mg gel strips.

Gel preparation for gentamicin and ketorolac delivery with chitosan nanoparticles

0-10 mg of ketorolac loaded chitosan nanoparticle powder and 0-10 mg of gentamicin powder was mixed in a vial with 250 mg of MA-PGA₂-PEG₉-PLA₂-MA of 10 mg/mL Phenylbis (2,4,6-trimethylbenzoyl) phosphine oxide initiator at a ratio of 1 μL each 20 μL of macromer and exposed to UV radiation at 395 nm for two minutes to form a 250 mg gel strips.

### Example 4

Sequential dual drug release from alginate chitosan nanoparticle doped MA-PLA₄-PEG₉-PLA₄-MA gels
Synthesis of MA-PLA₄-PEG₉-PLA₄-MA

3 g of PEG with a molecular weight of 400 g/mol was mixed with 4.32 g of DL lactide and 60 ul of stannous octoate. The mixture was heated with microwave radiation for 2 minutes to produce PLA₄-PEG₉-PLA₄. 2.5 ml of methacrylic anhydride was added and the mixture was heated with microwave radiation for 2 minutes to produce MA-PLA₄-PEG₉-PLA₄-MA. Later, the macromers were washed using hydrophobic solutions and hydrophilic solutions. 70 uL mequinol was added to the macromers.

Synthesis of vancomycin-loaded alginate chitosan nanoparticles

2 mL of 2.25 mg/mL calcium chloride dihydrate and 0-30 mg/ml vancomycin hydrochloride solution was added dropwise to 10 mL of the 1.5 mg/mL alginate solution at pH 5.1 on magnetic stirrer. The mixture was let to sit for an hour mixing at the highest stirring speed. Later 2 mL of 0.8 mg/mL chitosan in acetic acid at pH 5.4 was added dropwise to the mixture on magnetic stirrer and left for homogenization for 2 hours. The particles were washed three times at 20,000×g by discarding the supernatant and adding 5 ml of distilled water. After washing, particles were dispersed in water and froze at −20° C. The frozen particles were lyophilized at least for 24 hours to remove water.

Synthesis of gentamicin-loaded alginate chitosan nanoparticles

2 mL of 2.25 mg/mL calcium chloride dihydrate and 0-2 mg/ml solution was added dropwise to 10 mL of the 1.5 mg/mL alginate solution at pH 5.1 on magnetic stirrer. The mixture was let to sit for an hour mixing at the highest stirring speed. Later 2 mL of 0.8 mg/mL chitosan in acetic acid at pH 5.4 was added dropwise to the mixture on magnetic stirrer and left for homogenization for 2 hours. The particles were washed three times at 20,000×g by discarding the supernatant and adding 5 ml of distilled water. After washing, particles were dispersed in water and froze at −20° C. The frozen particles were lyophilized at least for 24 hours to remove water.

Gel preparation for dual drug delivery
Gel preparation for ketorolac and vancomycin delivery with alginate chitosan nanoparticles

0-10 mg of ketorolac (tromethamine) powder and 0-10 mg of vancomycin (hydrochloride)-loaded alginate chitosan nanoparticle powder was mixed in a vial with 250 mg of MA-PGA2-PEG9-PGA2-MA of 10 mg/mL Phenylbis (2,4,6-trimethylbenzoyl) phosphine oxide initiator at a ratio of 1μL each 20 μL of macromer and exposed to UV radiation at 395 nm for two minutes to form a 250 mg gel strips.

Gel preparation for ketorolac and gentamicin delivery with alginate chitosan nanoparticles

0-10 mg of ketorolac (tromethamine) powder and 0-10 mg of gentamicin (sulfate)-loaded alginate chitosan nanoparticle powder was mixed in a vial with 250 mg of MA-PGA₂-PEG₉-PGA₂-MA of 10 mg/mL Phenylbis (2,4,6-trimethylbenzoyl) phosphine oxide initiator at a ratio of 1 μL each 20 μL of macromer and exposed to UV radiation at 395 nm for two minutes to form a 250 mg gel strips.

### Example 5

Sequential dual drug release from alginate chitosan nanoparticle doped MA-PGA₂-PEG₉-PLA₂-MA gels
Synthesis of MA-PGA₂-PEG₉-PGA₂-MA

3 g of PEG with a molecular weight of 400 g/mol was mixed with 1.72 g of DL glycolide and 75 ul of stannous octoate. The mixture was heated with microwave radiation for 2 minutes to produce PLA₄-PEG₉-PLA₄. 2.5 ml of methacrylic anhydride was added and the mixture was heated with microwave radiation for 2 minutes to produce MA-PGA₂-PEG₉-PGA₂-MA. Later, the macromers were washed using hydrophobic solutions and hydrophilic solutions. 48 ul mequinol was added to the macromers.

Synthesis of vancomycin-loaded alginate chitosan nanoparticles

2 mL of 2.25 mg/mL calcium chloride dihydrate and 0-30 mg/ml vancomycin hydrochloride solution was added dropwise to 10 mL of the 1.5 mg/mL alginate solute ion at pH 5.1 on magnetic stirrer. The mixture was let to sit for an hour mixing at the highest stirring speed. Later 2 mL of 0.8 mg/mL chitosan in acetic acid at pH 5.4 was added dropwise to the mixture on magnetic stirrer and left for homogenization for 2 hours. The particles were washed three times at 20,000×g by discarding the supernatant and adding 5 ml of distilled water. After washing, particles were dispersed in water and froze at −20° C. The frozen particles were lyophilized at least for 24 hours to remove water.

Synthesis of gentamicin-loaded alginate chitosan nanoparticles

2 mL of 2.25 mg/mL calcium chloride dihydrate and 0-2 mg/ml gentamicin sulfate solution was added dropwise to 10 mL of the 1.5 mg/mL alginate solution at pH 5.1 on magnetic stirrer. The mixture was let to sit for an hour mixing at the highest stirring speed. Later 2 mL of 0.8 mg/mL chitosan in acetic acid at pH 5.4 was added dropwise to the mixture on magnetic stirrer and left for homogenization for 2 hours. The particles were washed three times at 20,000×g by discarding the supernatant and adding 5 ml of distilled water. After washing, particles were dispersed in water and froze at −20° C. The frozen particles were lyophilized at least for 24 hours to remove water.

Gel preparation for dual drug delivery
Gel preparation for ketorolac and vancomycin delivery with alginate chitosan nanoparticles

0-10 mg of ketorolac (tromethamine) powder and 0-10 mg of vancomycin (hydrochloride)-loaded alginate chitosan nanoparticle powder was mixed in a vial with 250 mg of MA-PLA₄-PEG₉-PLA₄-MA of 10 mg/mL Phenylbis (2,4,6-trimethylbenzoyl) phosphine oxide initiator at a ratio of 1 μL each 20 μL of macromer and exposed to UV radiation at 395 nm for two minutes to form a 250 mg gel strips.

Gel preparation for ketorolac and gentamicin delivery with alginate chitosan nanoparticles

0-10 mg of ketorolac (tromethamine) powder and 0-10 mg of gentamicin (sulfate)-loaded alginate chitosan nanoparticle powder was mixed in a vial with 250 mg of MA-PLA₄-PEG₉-PLA₄-MA of 10 mg/mL Phenylbis (2,4,6-trimethylbenzoyl) phosphine oxide initiator at a ratio of 1 μL each 20 μL of macromer and exposed to UV radiation at 395 nm for two minutes to form a 250 mg gel strips.

The following references are incorporated herein in their entirety:
[1] Lenguerrand et al. BMJ Open 2017; 7: e014056.
[2] Leong, et al., The Bone & Joint Journal 2020 102-B: 8, 997-1002
[3] Gil, et al., Diagnostic Microbiology and Infectious Disease, Vol 96, Iss4,2020;
[4] Gubernator, et al., International Journal of Pharmaceutics, Vol 327, Iss1-2, 2006, P104-109

What is claimed is:

1. A method of making a biodegradable composition for localized drug delivery, the method comprising:
polymerizing a mixture of biodegradable macromers, biopolymeric nanoparticles, an initiator, and one or more therapeutic agents,
wherein the one or more therapeutic agents are dispersed within the macromers, loaded within the biopolymeric nanoparticles, or both dispersed within the macromers and loaded within the biopolymeric nanoparticles.

2. The method according to claim 1, wherein the one or more therapeutic agents are dispersed within methacrylated polyethylene based biodegradable macromers.

3. The method according to claim 1, wherein the one or more therapeutic agents are loaded within the biopolymeric nanoparticles.

4. The method according to claim 1, wherein a first therapeutic agent is dispersed within methacrylated polyethylene based biodegradable macromers and a second therapeutic agent is loaded within the biopolymeric nanoparticles.

5. The method of any one of claims 1-4, wherein the mixture is polymerized on a medical device surface.

6. The method according to claim 5, wherein the mixture is polymerized on the medical device surface in situ.

7. The method according to claim 5, wherein the mixture is polymerized on the medical device surface at a site of implantation of the medical device.

8. The method according to claim 5, wherein the mixture is polymerized by irradiating the mixture with ultraviolet radiation.

9. The method according to any one of claims 1-4, wherein the macromers comprise a biodegradable moiety of repeated lactic acid units, repeated glycolic acid units, or both repeated lactic acid units and glycolic acid units and/or the biopolymeric nanoparticles comprise alginate and/or chitosan.

10. The method according to claim 9, wherein the macromers are methacrylated polyethylene based biodegradable macromers comprising a biodegradable moiety of repeated lactic acid units.

11. The method according to claim 9, wherein the macromers are methacrylated polyethylene based biodegradable macromers comprising a biodegradable moiety of repeated glycolic acid units.

12. The method according to claim 9, wherein the biopolymeric nanoparticles comprise alginate and/or chitosan.

13. The method according to claim 9, wherein—
the mixture comprises methacrylate-polylactic acid-polyethylene glycol-polylactic acid-methacrylate macromers, vancomycin dispersed within the methacrylate-polylactic acid-polyethylene glycol-polylactic acid-methacrylate macromers, and biopolymeric nanoparticles comprising chitosan and ketorolac loaded therein; or
the mixture comprises methacrylate-polylactic acid-polyethylene glycol-polylactic acid-methacrylate macromers, gentamicin dispersed within the methacrylate-polylactic acid-polyethylene glycol-polylactic acid-methacrylate macromers, and biopolymeric nanoparticles comprising chitosan and ketorolac loaded therein; or
the mixture comprises methacrylate-polylactic acid-polyethylene glycol-polylactic acid-methacrylate macromers, ketorolac dispersed with the methacrylate-polylactic acid-polyethylene glycol-polylactic acid-methacrylate macromers, and biopolymeric nanoparticles comprising alginate and chitosan and vancomycin loaded therein; or
the mixture comprises methacrylate-polylactic acid-polyethylene glycol-polylactic acid-methacrylate macromers, ketorolac dispersed with the methacrylate-polylactic acid-polyethylene glycol-polylactic acid-methacrylate macromers, and biopolymeric nanoparticles comprising alginate, chitosan and gentamicin loaded therein.

14. The method according to claim 13, wherein the macromers comprise four repeated polylactic acid units and a weight-averaged molecular weight of polyethylene glycol of 400 g/mol.

15. The method according to claim 9, wherein—
the mixture comprises methacrylate-polyglycolic acid-polyethylene glycol-polyglycolic acid-methacrylate macromers, vancomycin dispersed with the methacrylate-polyglycolic acid-polyethylene glycol-polyglycolic acid-methacrylate macromers, and biopolymeric nanoparticles comprising chitosan, and ketorolac loaded therein; or
the mixture comprises methacrylate-polyglycolic acid-polyethylene glycol-polyglycolic acid-methacrylate macromers, gentamicin dispersed with the methacrylate-polyglycolic acid-polyethylene glycol-polyglycolic acid-methacrylate macromers, and biopolymeric nanoparticles comprising chitosan, and ketorolac loaded therein; or
the mixture comprises methacrylate-polyglycolic acid-polyethylene glycol-polyglycolic acid-methacrylate macromers, ketorolac dispersed with the methacrylate-polyglycolic acid-polyethylene glycol-polyglycolic acid-methacrylate macromers, and biopolymeric nanoparticles comprising alginate and chitosan, and vancomycin loaded therein; or
the mixture comprises methacrylate-polyglycolic acid-polyethylene glycol-polyglycolic acid-methacrylate macromers, ketorolac dispersed with the methacrylate-polyglycolic acid-polyethylene glycol-polyglycolic acid-methacrylate macromers, and biopolymeric nanoparticles comprising alginate and chitosan, and gentamicin loaded therein.

16. The method according to claim 15, wherein the macromers include two repeated polyglycolic acid units and a weight-averaged molecular weight of polyethylene glycol is 200 g/mol.

17. The method of any one of claims 10-16, wherein the mixture is polymerized on a medical device surface.

18. The method according to claim 17, wherein the mixture is polymerized on the medical device surface in situ.

19. The method according to claim 17, wherein the mixture is polymerized on the medical device surface at a site of implantation of the medical device.

20. The method according to claim 17, wherein the mixture is polymerized by irradiating the mixture with ultraviolet radiation.

21. A kit for preparing a biodegradable composition for localized drug delivery, the kit comprising:
a first container having biodegradable macromers therein;
a second container having biopolymeric nanoparticles therein; and
a third container having an initiator therein.

22. The kit according to claim 21, further comprising one or more therapeutic agents, wherein the one or more therapeutic agents are dispersed within the macromers, loaded within the biopolymeric nanoparticles, or both dispersed within the macromers and loaded within the biopolymeric nanoparticles.

23. The kit according to claim 22, wherein the one or more therapeutic agents are dispersed within methacrylated polyethylene based biodegradable macromers.

24. The kit according to claim 22, wherein the one or more therapeutic agents are loaded in the plurality of biopolymeric nanoparticles.

25. The kit according to claim 22, wherein a first therapeutic agent is dispersed within the methacrylated polyethylene based biodegradable macromers and a second therapeutic agent is loaded within the biopolymeric nanoparticles.

26. The kit according to claim 22 further comprising a fourth container having the one or more therapeutic agents therein.

27. The kit according to claim 26, wherein a first therapeutic agent is contained in the fourth container and a second therapeutic agent is loaded within the biopolymeric nanoparticles.

28. The kit according to any one of claims 21-27 further comprising:
an applicator configured to apply a mixture of the biodegradable macromers; the biopolymeric nanoparticles, the initiator, and one or more therapeutic agents to a medical device surface; or
a medical device; or
a radiation source; or
any combination thereof.

29. The kit according to any one of claims 21-28, wherein the first container, the second container, and the third container are different containers.

30. The kit according to any one of claims 21-28, wherein the first containing and the second container are the same container and the third container is a different container.

31. A method of preparing a biodegradable composition for localized drug delivery, the method comprising:
mixing the contents of the first container, the second container, and the third container according to the kit of claim 21
polymerizing a mixture,
wherein the mixture comprises one or more therapeutic agents dispersed within the biodegradable macromers, loaded within the biopolymeric nanoparticles, or both dispersed within the biodegradable macromers and loaded within the biopolymeric nanoparticles.

32. The method according to claim 31, wherein the one or more therapeutic agents are dispersed within methacrylated polyethylene based biodegradable macromers.

33. The method according to claim 31, wherein the one or more therapeutic agents are loaded in the plurality of biopolymeric nanoparticles.

34. The method according to claim 31, wherein a first therapeutic agent is dispersed within methacrylated polyethylene based biodegradable macromers and a second therapeutic agent is loaded within the biopolymeric nanoparticles.

35. The method according to claim 31, further comprising mixing the contents of a fourth container having the one or more therapeutic agents therein with the contents of the first container, the second container, and the third container.

36. The method according to claim 35, wherein a first therapeutic agent is contained in the fourth container and a second therapeutic agent is loaded within the biopolymeric nanoparticles.

37. The method according to any one of claims 31-36, wherein the mixture is applied to a medical device surface prior to polymerization of the mixture.

38. The method of claim 37, wherein the mixture is applied to the medical device surface in situ.

39. The method of claim 37, wherein the mixture is applied to the medical device surface at a site of implantation of the medical device.

40. The method of claim 37, wherein the mixture is polymerized by irradiating the mixture with ultraviolet radiation.

41. A mixture comprising:
biodegradable macromers; biopolymeric nanoparticles, an initiator, and one or more therapeutic agents, wherein the one or more therapeutic agents are dispersed within the macromers, loaded within the biopolymeric nanoparticles, or both dispersed within the biodegradable macromers and loaded within the biopolymeric nanoparticle.

42. A medical device having the mixture according to claim 41 thereon.

43. A biodegradable composition for localized delivery comprising a polymerized mixture according to claim 41.

44. A biodegradable composition for localized delivery prepared according to any one of the preceding claims.

45. A medical device having the biodegradable composition for localized delivery according to claim 44 thereon.

46. A medical device having the biodegradable composition for localized delivery prepared according to any one of the preceding claims thereon.
